(19) United States
(12) Patent Application Publication
(10) Pub. No.: US 2024/0282451 A1
(43) Pub. Date: Aug. 22, 2024

(54) ARTIFICIAL INTELLIGENCE TO GENERATE LABOR AND DELIVERY PREDICTIONS

(71) Applicant: GE Precision Healthcare LLC, Waukesha, WI (US)

(72) Inventors: Rohit Pardasani, Bengaluru (IN); Siddharth Ajith, Bengaluru (IN); John Michael Jordan, Severna Park, MD (US)

(21) Appl. No.: 18/631,749

(22) Filed: Apr. 10, 2024

Related U.S. Application Data
(63) Continuation-in-part of application No. 17/517,251, filed on Nov. 2, 2021.

(51) Int. Cl. G16H 50/20; A61B 5/00; A61B 5/0205; G16H 50/30

(52) U.S. Cl. CPC G16H 50/20; A61B 5/0205; A61B 5/4362; G16H 50/30

(57) ABSTRACT

One or more systems, devices, computer-implemented methods and/or computer program products of use provided herein relate to artificial intelligence (AI) to generate labor and delivery-based predictions. A system can comprise a processor that can execute computer-executable components stored in memory, wherein the computer-executable components can comprise a first AI model that can generate first data comprising one or more labor and delivery predictions applicable to one or more fetuses and a mother of the one or more fetuses, during labor, by analyzing second data comprising cardiotocography (CTG) analysis data of the one or more fetuses and the mother generated by a second AI model and third data comprising maternal health analysis data of the mother generated by a third AI model, wherein the first AI model can be a multistage AI model comprising respective models directed to predicting respective ones of the one or more labor and delivery predictions.

### CROSS-REFERENCE TO RELATED APPLICATIONS

This application is a Continuation-In-Part of and claims priority to U.S. patent application Ser. No. 17/517,251 filed on Nov. 2, 2021, entitled “DEEP LEARNING BASED FETAL HEART RATE ANALYTICS.” The entirety of the aforementioned application is incorporated by reference herein.

### TECHNICAL FIELD

The subject disclosure generally relates to artificial intelligence (AI), and more specifically, to an AI-based approach to generate labor and delivery-based clinical predictions.

### BACKGROUND

Discrete algorithms, systems, and techniques are often employed to analyze portions of clinical data regarding the health of a fetus and that of the mother of the fetus to generate clinical predictions; however, such clinical predictions are often inadequate by themselves to assist clinicians in making decisions regarding near term or immediate actions. Moreover, such algorithms, systems and techniques often do not analyze certain clinical parameters, which can have an adverse impact on clinicians' decision making.

The above-described background description is merely intended to provide a contextual overview regarding fetal monitoring and maternal monitoring techniques and is not intended to be exhaustive.

### SUMMARY

The following presents a summary to provide a basic understanding of one or more embodiments of the invention. This summary is not intended to identify key or critical elements or delineate any scope of the different embodiments or any scope of the claims. Its sole purpose is to present concepts in a simplified form as a prelude to the more detailed description that is presented later.

According to an embodiment, a system is provided. The system can comprise a processor that can execute computer-executable components stored in memory, wherein the computer-executable components can comprise a first AI model that can generate first data comprising one or more labor and delivery predictions applicable to one or more fetuses and a mother of the one or more fetuses, during labor, by analyzing second data comprising cardiotocography (CTG) analysis data of the one or more fetuses and the mother generated by a second AI model and third data comprising maternal health analysis data of the mother generated by a third AI model, wherein the first AI model can be a multistage AI model comprising respective models directed to predicting respective ones of the one or more labor and delivery predictions.

In some embodiments, elements described in the disclosed systems and methods can be embodied in different forms such as a computer-implemented method, a computer program product, or another form.

### DESCRIPTION OF THE DRAWINGS

FIG. 1 illustrates a block diagram of an example, non-limiting system that can generate labor and delivery predictions and facilitate fetal heart rate (FHR) analytics by employing machine learning techniques in accordance with one or more embodiments of the disclosed subject matter.

FIG. 2 illustrates a flow diagram of an example, non-limiting method that can generate labor and delivery predictions by analyzing CTG analysis data and maternal health analysis data in accordance with one or more embodiments of the disclosed subject matter.

FIG. 3 illustrates a flow diagram of an example, non-limiting method that can generate an estimate of fetal potential of hydrogen (pH) and predict the probability of fetal acidemia by analyzing CTG analysis data and maternal health analysis data in accordance with one or more embodiments of the disclosed subject matter.

FIG. 4 illustrates an example, non-limiting graph of a labor curve in accordance with one or more embodiments of the disclosed subject matter.

FIG. 5 illustrates an example, non-limiting graph of a fetal pH curve in accordance with one or more embodiments of the disclosed subject matter.

FIG. 6 illustrates a flow diagram of an example, non-limiting method that can generate a recommendation for a C-section in accordance with one or more embodiments of the disclosed subject matter.

FIG. 7 illustrates an example, non-limiting graph based of FHR variability in accordance with one or more embodiments of the disclosed subject matter.

FIG. 8 presents example FHR and UA tracings in accordance with one or more embodiments of the disclosed subject matter.

FIG. 9 presents example FHR and UA tracings comparing the model identified patterns with the ground truth annotated patterns in accordance with one or more embodiments of the disclosed subject matter.

FIG. 10 presents additional example FHR and UA tracings comparing the model identified patterns with the ground truth annotated patterns in accordance with one or more embodiments of the disclosed subject matter.

FIG. 11 presents an example training process for training a machine learning model to detect defined patterns in cardiograph data in accordance with one or more embodiments of the disclosed subject matter.

FIG. 12 presents a flow-diagram of an example process for performing FHR-UA analytics in real-time by employing machine learning techniques in accordance with one or more embodiments of the disclosed subject matter.

FIG. 13 illustrates an example system for performing FHR-UA analytics in real-time by employing machine learning techniques in accordance with one or more embodiments of the disclosed subject matter.

FIG. 14 presents a high-level flow diagram of an example computer-implemented process to perform FHR analytics by employing machine learning techniques in accordance with one or more embodiments of the disclosed subject matter.

FIG. 15 presents a high-level flow diagram of another example computer-implemented process to perform FHR analytics by employing machine learning techniques in accordance with one or more embodiments of the disclosed subject matter.

FIG. 16 illustrates a flow diagram of an example, non-limiting method that can generate labor and delivery predictions in accordance with one or more embodiments of the disclosed subject matter.

FIG. 17 illustrates a block diagram of an example, non-limiting operating environment in which one or more embodiments described herein can be facilitated.

### DETAILED DESCRIPTION

The following detailed description is merely illustrative and is not intended to limit embodiments and/or application or uses of embodiments. Furthermore, there is no intention to be bound by any expressed or implied information presented in the preceding Background section, Summary section or in the Detailed Description section.

One or more embodiments are now described with reference to the drawings, wherein like referenced numerals are used to refer to like elements throughout. In the following description, for purposes of explanation, numerous specific details are set forth in order to provide a more thorough understanding of the one or more embodiments. It is evident, however, in various cases, that the one or more embodiments can be practiced without these specific details.

### Definitions

Labor: A series of continuous progressive contractions of the uterus that cause the cervix to dilate and efface.

Cesarean (C)-section: Surgical delivery of a baby through an incision made in the mother's abdomen and uterus.

Dystocia: A challenging birth resulting from an awkwardly positioned fetus, a small maternal pelvis or an inability of the uterus and cervix to contract normally.

Fetal hypoxia: A condition where the oxygen to the fetus is reduced, resulting in impaired fetal development and increased risk of fetal or infant mortality.

Fetal acidemia: A state of low blood Ph in a fetus's blood.

Fetal acidosis: Presence of high levels of acid in a fetus's blood, resulting from oxygen deprivation over an extended period of time. Acidosis is a process that causes acidemia.

Preeclampsia: A complicated pregnancy related condition that is typically characterized by high blood pressure, excess protein in urine, swelling.

Sepsis: A serious medical condition where the body reacts improperly to an infection.

Potential of hydrogen (pH): A measure of concentration of Hydrogen ions in a substance.

Numerous techniques and technologies are available to analyze and interpret FHR data to calculate fetal baseline heart rate (HR), maternal HR data, and other fetal health related parameters. However, such techniques are available as individual systems that can ingest various inputs and analyze the inputs to generate specific outputs, as opposed to a single system that can provide clinical decision support to clinicians attending to labor and delivery to assist the clinicians with key decisions to be made. For example, clinical views, information, and/or applications associated with different clinical parameters are often part of discrete systems having independent calculations and logic. For example, some existing AI based algorithmic systems can require inputs from multiple applications to execute their functions. Moreover, such systems or sub-systems often do not analyze certain clinical parameters, which can have an adverse impact on clinicians' decision making. The data analysis in existing systems is either only rule-based or based on a limited set of data items and parameters. Although discrete algorithms, systems, and techniques can be employed to analyze a portion of the information or clinical data regarding the health of the fetus and the mother to generate predictions, such inputs by themselves are often inadequate in assisting a clinician to make decisions regarding near term or immediate actions. For example, in surveillance and documentation systems based on fetal and uterine activity parameters (e.g., PeriWatch Surveillance™, Centricity™ Perinatal) input data is limited to FHR and UA, and FHR and UA related events. In another example, systems that can generate patient safety alerts based on fetal and maternal data analysis (e.g., PeriGen® Laborwatch) are manual systems that are entirely dependent on clinicians' opinions on the data available, and do not perform any intelligence on data. In yet another example, an early warning system based on fetal distress (e.g., Periwatch Vigilance) is an automated intelligent system based on limited input parameters from electronic medical records (EMRs) or the surveillance and documentation system mentioned supra.

Embodiments described herein include systems, apparatus, computer-implemented methods and/or computer program products that can employ AI and heuristics-based algorithms to analyze various clinical input parameters and recommend clinical actions and treatment pathways during a period of labor of a patient. Such clinical actions and treatment pathways can be employed by one or more entities to reduce the risks involved during labor and delivery for a patient. In some embodiments, the entities can be human entities such as labor and delivery (L and D) clinicians, L and D nurses and obstetrics physicians. In other embodiments, the entities can be hardware, software, AI, neural network, and/or machine.

In an embodiment, an integrated system is provided that can process clinical inputs to generate further clinical data, have a mathematical capability for analysis of the clinical data generated, and have the capability to present an integrated view of a final clinical output that can assist medical professionals with clinical decision support during periods of labor and delivery of a patient. For example, in an embodiment, a first AI model can generate first data comprising one or more labor and delivery predictions applicable to one or more fetuses and a mother of the one or more fetuses, during labor, by analyzing second data comprising CTG analysis data of the one or more fetuses and the mother generated by a second AI model and third data comprising maternal health analysis data of the mother generated by a third AI model, wherein the first AI model can be a multistage AI model comprising respective models directed to predicting respective ones of the one or more labor and delivery predictions.

In an embodiment, the second AI model and the third AI model can perform a first level of processing wherein the second AI model and the third AI model can ingest numerical data, time series data and/or waveform data and perform AI-based and heuristics-based analyses on the ingested data to generate the second data and the third data, and the first AI model can ingest the second data and the third data to generate the first data by performing a second level of processing. The data ingested by the second AI model and the third AI model can be generated by devices (e.g., a fetal monitoring system (FMS), a cardiotocograph, etc.) employed to perform fetal monitoring and maternal monitoring during a period of labor of a pregnant woman and from EMRs that can be generated prior to and during the labor. In an embodiment, the first AI model can comprise a long short-term memory (LSTM) model, a machine learning regression model, and/or one or more additional models dedicated to generating respective ones of the one or more labor and delivery predictions. In an embodiment, the second AI model can be a cardiotocograph pattern identification model.

Such embodiments can provide a rule-based system guided by published clinical standards and guidelines and AI driven insights that can be trained by employing data annotated according to clinicians' experiences. The systems disclosed herein can provide benefits to clinicians and patients via a descriptive analysis of fetal strips with events and parameters highlighted, alerts and alarms for actions to be executed, and an integrated analysis and views or graphical depictions of maternal health parameters and fetal health parameters. Further, the systems disclosed herein can provide AI models that can generate clinical outputs and alerts, and the systems can have the ability to review and analyze a 360 degree view of patient data to provide labor and delivery related insights.

Embodiments of the disclosed subject matter are further directed to systems, apparatus, computer-implemented methods and/or computer program products that can facilitate automated interpretation and analysis of patient cardiotocograph data in real-time, particularly in the context of labor and delivery. CTG records changes in the FHR and its temporal relationship to uterine contractions. The machine used to perform the monitoring is called a cardiotocograph, more commonly known as an electronic fetal monitor (EFM). Simultaneous recordings are performed by two separate transducers, one for the measurement of the FHR and a second one for the uterine contractions, also referred to as the uterine activity (UA). FHR and UA tracings are typically analyzed manually by an obstetric medical team (e.g., including nurses, resident physicians, nurse midwives, attending physicians, etc.) over the course of labor to identify abnormal patterns. Presently there are multiple challenges in analysis of FHR-UA tracings. The manual visual assessment of the graphical tracings requires expertise and may be biased due to the level of experience of the physician/nurse performing the analysis. Although some automated methods for assessing FHR-UA tracings have been developed, these methods are based solely on guidelines and definitions for identifying patterns and conditions expressed in terms of mathematical rules. The existing computational/algorithmic methods of assessment are thus strictly statistical, and rule based. This automatic assessment is often not in accordance with the analysis based on experience of clinicians. Most experts feel that going strictly by these rule-based definitions is not appropriate in real life scenarios. Thus, there is a need for a computational method for analysis of FHR-UA tracings which is not solely rule-based and assimilates the visual assessment expertise of clinicians in real-life scenarios.

In this regard, the disclosed techniques can solve the challenge of automatic analysis of FHR and UA tracings by employing a data driven approach rooted in machine learning as opposed to a solely rule-based approach. This data driven approach can involve training one or more machine learning models by employing a supervised machine learning process to identify distinct patterns in cardiotocograph data that correspond to defined physiological events associated with the fetus and/or the mother. For example, the defined physiological events can include, but are not limited to, an FHR acceleration, an FHR deceleration, a period of FHR variability and a contraction. Once trained, the one or more pattern recognition models can be deployed in real-life settings in inferencing modes to automatically detect occurrences of the defined physiological events based on cardiograph data captured for a mother and fetus in real-time. The training data used to train the one or more pattern recognition models can comprise cardiograph data annotated with information identifying the patterns and their corresponding physiological events. The subjects represented in the training data can include a plurality of different mothers and their fetuses as monitored over their course of labor. This data driven approach can ensure that the inferences generated by the pattern recognition models are based on learning from analysis of actual cardiograph data captured for real patients and can, thus, mimic the visual FHR-UA strip analysis of experts in actual clinical contexts.

In an embodiment, the one or more trained cardiotocograph pattern recognition models can be integrated into software and/or hardware products that involve FHR monitoring and diagnosis. The trained models can be employed to evaluate both static and streaming cardiotocograph data. For example, the one or more models can be employed to retrospectively analyze recorded cardiograph data captured for a subject (e.g., mother and fetus) during a monitoring session. The one or more models can also be employed to analyze a continuous stream of cardiotocograph data flowing from a cardiotocograph device attached to a mother and fetus in real-time. Regardless of the deployment scenario, the trained cardiotocograph pattern recognition model can drastically reduce the manual strip assessment time while minimizing errors.

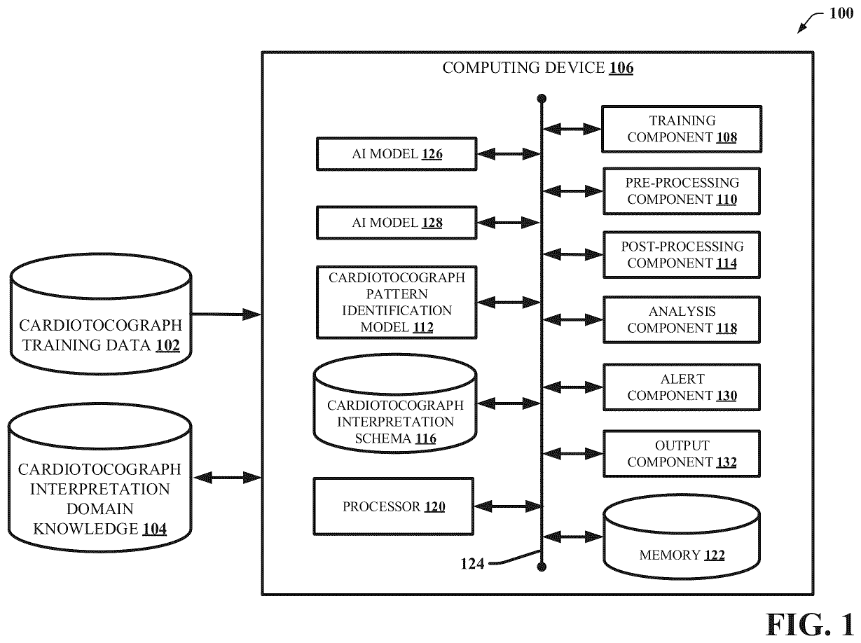

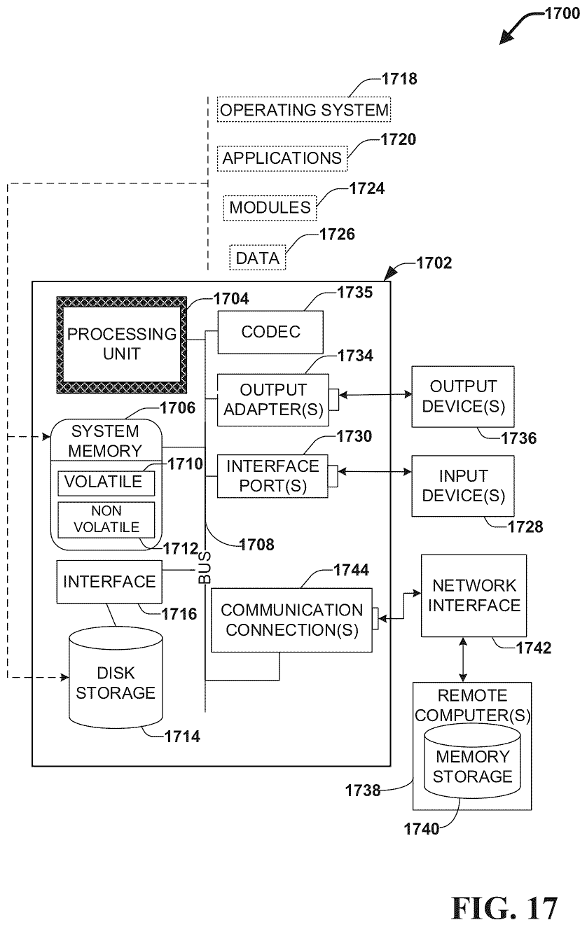

The embodiments depicted in one or more figures described herein are for illustration only, and as such, the architecture of embodiments is not limited to the systems, devices and/or components depicted therein, nor to any particular order, connection and/or coupling of systems, devices and/or components depicted therein. For example, in one or more embodiments, the non-limiting systems described herein, such as non-limiting system 100 as illustrated at FIG. 1, and/or systems thereof, can further comprise, be associated with and/or be coupled to one or more computer and/or computing-based elements described herein with reference to an operating environment, such as the operating environment 1700 illustrated at FIG. 17. For example, non-limiting system 100 can be associated with, such as accessible via, a computing environment 1700 described below with reference to FIG. 17, such that aspects of processing can be distributed between non-limiting system 100 and the computing environment 1700. In one or more described embodiments, computer and/or computing-based elements can be used in connection with implementing one or more of the systems, devices, components and/or computer-implemented operations shown and/or described in connection with FIG. 1 and/or with other figures described herein.

FIG. 1 illustrates a block diagram of an example, non-limiting system 100 that can generate labor and delivery predictions and facilitate FHR analytics by employing machine learning techniques in accordance with one or more embodiments of the disclosed subject matter. Embodiments of systems described herein can include one or more machine-executable components embodied within one or more machines (e.g., embodied in one or more computer-readable storage media associated with one or more machines). Such components, when executed by the one or more machines (e.g., processors, computers, computing devices, virtual machines, etc.) can cause the one or more machines to perform the operations described.

In this regard, non-limiting system 100 can comprise computing device 106 that can comprise several computer-executable components. These computer-executable components can include training component 108, pre-processing component 110, cardiotocograph pattern identification model 112, post-processing component 114, analysis component 118, AI model 126, AI model 128, alert component 130, and/or output component 132. These computer/machine executable components (and others described herein) can be stored in memory associated with the one or more machines (e.g., computing device 106). The type of computing device 106 can vary. For example, computing device 106 can correspond to a personal computing device (e.g., a desktop computer, a laptop computer, a smartphone, etc.), a server device, a mobile device, a virtual device, and so on. The memory can further be operatively coupled to at least one processor, such that the components can be executed by the at least one processor to perform the operations described. For example, in some embodiments, the computer/machine-executable components can be stored in memory 122 that can be coupled to processor 120 for execution thereof. Examples of memory 122 and processor 120 as well as other suitable computer or computing-based elements, can be found with reference to FIG. 17, and can be employed in connection with implementing one or more of the systems or components shown and described in connection with FIG. 1 or other figures disclosed herein. Computing device 106 can further comprise device bus 124 that can communicatively and operatively couple the various components and elements of computing device 106 to one another.

In an embodiment, AI model 126 can generate first data comprising one or more labor and delivery predictions applicable to one or more fetuses and a mother of the one or more fetuses, during labor of the mother, by analyzing second data comprising CTG analysis data of the one or more fetuses and the mother generated by AI model 128 and third data comprising maternal health analysis data of the mother generated by cardiotocograph pattern identification model 112. For example, hypertension combined with other clinical factors can be good indicators of acidosis during labor, and AI model 126 can analyze multiple clinical factors to confirm such a hypothesis and generate a prediction with a confidence interval based on the hypothesis. In an embodiment, cardiotocograph pattern identification model 112 and AI model 128 can perform a first level of processing wherein cardiotocograph pattern identification model 112 and AI model 128 can ingest numerical data, time series data and/or waveform data and perform AI-based and heuristics-based analyses on the ingested data to respectively generate the second data and the third data, and AI model 126 can ingest the second data and the third data to generate the first data by performing a second level of processing. The first data can comprise one or more types of data selected from a group comprising fetal hypoxia, fetal acidemia, fetal acidosis, labor progression indicating a C-section, cervical dilation progression, a postpartum hemorrhage risk assessment, a preeclampsia prediction, a labor induction recommendation, a sepsis possibility, and one or more additional labor and delivery predictions.

In an embodiment, AI model 126 can be a multistage AI model comprising respective models directed to predicting respective ones of the one or more labor and delivery predictions. For example, AI model 126 can comprise an LSTM model, a machine learning regression model, and/or one or more additional AI and machine learning-based models that can be respectively dedicated to generating respective ones of the one or more labor and delivery predictions. In this regard, AI model 126 can also employ standard machine learning methods such as, for example, logistic regression, support vector machines (SVM), decision trees, etc. depending on the respective labor and delivery predictions. For example, some predictions generated by AI model 126 can include binary outcomes (e.g., “yes” and “no” outcomes) and a logistic regression framework can be suitable to generate such binary outcomes. Some examples of binary outcomes can include a prediction for whether a C-section is needed in a particular labor scenario, hypoxia predictions, etc. On the contrary, other predictions generated by AI model 126 can be multiclass predictions as opposed to binary predictions, and AI model 126 can employ suitable machine learning techniques to generate such predictions.

In an embodiment, the data ingested by cardiotocograph pattern identification model 112 and AI model 128 can be generated by devices employed to perform fetal monitoring and maternal monitoring during a period of labor of a pregnant woman and from EMRs that can be generated prior to and/or during the labor. In this regard, the second data and the third data can be generated during fetal monitoring of the one or more fetuses and maternal monitoring of the mother, and the second data and the third data can be available for analysis by AI model 126 during the fetal monitoring of the one or more fetuses and the maternal monitoring of the mother. More specifically, during labor, clinically relevant information related to the mother and the one or more fetuses can be generated via one or more devices employed for fetal monitoring and maternal monitoring. The one or more devices referenced herein can comprise an FMS, a cardiotocograph, internal or external leads such as temperature of SpO2 leads, sensors, probes, etc., and the information captured by the one or more devices can include values computed by the one or more devices, for example, in the patient room of a hospital, clinic or other medical facility during the period of labor. In addition to data from the one or more devices (e.g., FMS, CTG, etc.), EMR information and parameters captured via laboratory tests, check-ups by clinicians, notes generated by nurses or other entities (e.g., hardware, software, AI, neural network, machine, and/or user), and so on, can be input to computing device 106. Such data can be accessed by cardiotocograph pattern identification model 112 and AI model 128 to respectively generate the second data and the third data. As such, patient parameters can be captured and employed for a first level of assessment. Thereafter, AI model 126 can employ the second data and the third data for a second level of assessment to generate the one or more labor and delivery predictions.

Fetal monitoring can capture clinical information about the one or more fetuses as well as the HR of the mother and other vital parameters associated with the mother, whereas maternal monitoring can capture more detailed clinical parameters associated with the mother and some clinical parameters associated with the one or more fetuses. The vital parameters can include HR, respiratory rate (RR), temperature, blood pressure, and SpO2 information, wherein the SpO2 information for an individual can be indicative of the amount of oxygen in the individual's blood. In some embodiments, the mother's temperature can be captured separately via a lead and documented as an EMR parameter. For example, unless the mother has a fever, the mother's body temperature is captured infrequently via a handheld device and documented. In other embodiments, the fetal monitor or the FMS employed to monitor the one or more fetuses can also be employed to capture the mother's temperature. In general, EMR data can be generated via documentation whereas other relevant clinical data can be generated via fetal and/or maternal monitoring. Thus, fetal monitoring can capture fetal parameters as well as maternal parameters.

As stated supra, cardiotocograph pattern identification model 112 and AI model 128 can ingest numerical data, time series data and/or waveform data and perform AI-based and heuristics-based analyses on the ingested data to respectively generate the second data and the third data. For example, cardiotocograph pattern identification model 112 can perform CTG analysis of waveform data comprising FHR data of the one or more fetuses and UA data of the mother to generate the second data, and AI model 128 can perform maternal health analysis of numerical data or time series data comprising other clinical information associated with mother and the one or more fetuses. Much of the input data utilized by AI model 128 to generate the third data can comprise clinical parameters of the mother, and some clinical parameters from the one or more fetuses. In an embodiment, the numerical data employed by AI model 128 can also be graphed. Some of the input data utilized by AI model 128 can be high frequency data, whereas other data can be low frequency data. For example, some input data can be collected more frequently, such as once every hour during labor, whereas other input data can be collected less frequently, such as once every day. The different types of input data employed by cardiotocograph pattern identification model 112 and AI model 128 can be time synchronized by pre-processing component 110 to structure the input data for cardiotocograph pattern identification model 112 and AI model 128.

In some embodiments, the numerical or time series data employed by AI model 128 can be labelled for the mother and be labelled with time stamps to align the time series data with the input data employed by cardiotocograph pattern identification model 112. In general, cardiotocograph pattern identification model 112 and AI model 128 can employ raw temporal data to respectively generate the second data and the third data, and AI model 126 can employ a variety of algorithms to process the second data and the third data to generate the first data. In some scenarios, AI model 126 can employ feature learning-based machine learning algorithms that do not need high frequency data, etc. In other scenarios, AI model 126 can employ classical machine learning algorithms to generate the first data since the second data and the third data are not high-fidelity data as opposed to, for example, waveforms processed by cardiotocograph pattern identification model 112 that are high-fidelity data and can often not be processed via classical machine learning algorithms.

In an embodiment, cardiotocograph pattern identification model 112 can generate the second data by processing FHR data of the one or more fetuses and UA data of the mother, and the second data can comprise one or more types of data selected from a group consisting of an FHR baseline value calculation, an FHR acceleration, an FHR deceleration, a contraction, an FHR variability value calculation, fetal tracing classification, and one or more additional CTG analysis data types. Similarly, AI model 128 can generate the third data by employing rule-based algorithms to process EMRs and health parameters of the mother, and the third data can comprise one or more types of data selected from a group consisting of maternal health related risk factors, pregnancy related complications, dystocia, genetic disorders and one or more additional maternal health analysis data types.

In an embodiment, the second data generated by cardiotocograph pattern identification model 112, and the third data generated by AI model 128 can be leveraged to train AI model 126. For example, training component 108 can train AI model 126 to generate the first data, by employing data features similar to those comprised in the second data and the third data as input data to AI model 126 and employing known labor and delivery predictions corresponding to the input data as output data for AI model 126. For example, training component 108 can employ training data comprising CTG analysis features such as FHR baseline value calculations, FHR acceleration, deceleration, and contraction identification, FHR variability value calculations, fetal tracing classifications, and one or more additional CTG analysis features, and maternal health analysis features such as maternal health related risk factors, pregnancy related complications, dystocia, genetic disorders and one or more additional maternal health analysis features as the input data to train AI model 126. The training data can further comprise ground truth data comprising patient information such as known clinical outcomes and predictions associated with respective CTG analysis features and maternal health analysis features. The ground truth information in the training data can comprise annotated/labelled data, such as, for example, labels provided by experienced clinicians and indicating presence of a fetal hypoxia condition at the time of a C-section performed on a patient, presence of acidemia, pH values of umbilical cords, etc.

Training component 108 can train AI model 126 to identify patterns of clinical outcomes by learning the relationships of the CTG analysis data and the maternal health analysis data to the corresponding known clinical outcomes and predictions. For example, the ground truth data can comprise characteristics related to fetal tracings and categorizations of the fetal tracings of certain clinical events and patterns (Y values) associated with the fetal tracing classifications generated by cardiotocograph pattern identification model 112 (X values). Training component 108 can employ the fetal tracing classifications as input data to train AI model 126 to predict the corresponding fetal tracing information from the ground truth data based on feature vectors associated with the fetal tracing classifications. Thus, the training data can comprise information about patterns that can be employed by training component 108 to train AI model 126 to make decisions about various medical conditions. In this manner, AI model 126 can be trained to predict fetal hypoxia, fetal acidemia, fetal acidosis, labor progression indicating a C-section, cervical dilation progression, a postpartum hemorrhage risk assessment, a preeclampsia prediction, a labor induction recommendation, a sepsis possibility, and one or more additional labor and delivery predictions.

In an embodiment, AI model 126 can be tested for accuracy of generating predictions (e.g., labor and delivery predictions such as hypoxia, acidosis, etc.) by comparing the predictions against known clinical outcomes in patient data previously recorded for a plurality of mothers and their respective fetuses, which known clinical outcomes can be comprised in the ground truth data. For example, after delivery, a blood oxygen saturation test can be performed in a laboratory for complicated delivery scenarios and the value can be recorded for such a test, venous or arterial pressure can be recorded, SpO2 saturation of the blood in the umbilical cord, which can be an excellent indicator of a fetus's oxygenation throughout delivery, can also be recorded. Such and other types of data from the ground truth data can be employed to test AI model 126.

In an embodiment, training component 108 can also train cardiotocograph pattern identification model 112 by employing a supervised machine learning process to identify patterns in training cardiotocograph data that correspond to defined physiological events associated with respective fetuses and mothers of the fetuses represented in the training cardiotocograph data. At least some of the training cardiotocograph data can comprise annotated cardiotocograph data annotated with information identifying the patterns and the defined physiological events that respectively correspond to the patterns, and the supervised machine learning process can comprise employing the annotated cardiotocograph data as ground truth. Additional embodiments related to cardiotocograph pattern identification model 112 are described in greater detail infra.

In an embodiment, output component 132 can display the first data generated by AI model 126 at computing device 106, wherein computing device 106 can be accessible to an entity (e.g., hardware, software, AI, neural network, machine and/or user) accessible to an entity for further analysis of the first data to identify actions to be executed by the entity for safe delivery of the one or more fetuses. In some embodiments, the first data can be output at a different device (e.g., a computer, a mobile phone, a tablet, etc.) accessible to the entity. In an embodiment, alert component 130 can generate an alert in response to the first data being indicative of an emergency situation. For example, alert component 130 can generate an alert if fetal pH values drop below a threshold value. Alert component 130 can generate the alert at the device accessible to the entity, and the entity can execute appropriate actions to manage such situations and ensure safe labor and delivery conditions for the mother and the one or more fetuses.

In addition to the computer-executable components of computing device 106, non-limiting system 100 can further comprise various data structures (e.g., data stores, databases, data files, and the like) that can provide information employed by the computer-executable components illustrated in FIG. 1 (and other figures described herein). This information can comprise cardiotocograph training data 102, cardiotocograph interpretation domain knowledge 104 and cardiotocograph interpretation schema 116. The type of data structures and/or data sources in which this information can be stored and located can vary. Computing device 106 and/or one or more components of computing device 106 can be operatively and communicatively coupled to these data structures either directly or via one or more wired or wireless communication networks (e.g., the Internet, an Intranet, etc.).

In an embodiment, training component 108 can provide for training and developing one or more machine learning models to identify one or more distinct patterns in cardiotocograph data that correspond to one or more defined physiological events associated with a fetus and/or mother from which the cardiotocograph data was captured. In the embodiment shown, these one or more machine learning models can correspond to cardiotocograph pattern identification model 112. Although a single model is illustrated, it should be appreciated that the number of cardiotocograph pattern identification models 112 can vary. For example, in some implementations, training component 108 can train a single model to identify different patterns corresponding to different physiological events. In other implementations, training component 108 can train separate models to identify different patterns corresponding to different physiological events. In other implementations, training component 108 can train separate models for different types of patients and/or patient groups. For example, the different types of patients or patient groups can be defined based on attributes associated with the mother and/or the fetus, including demographic attributes (e.g., age, gender body mass index (BMI), height/weight, location), clinical factors (e.g., comorbidities, risk level, pathology, etc.), medical history factors (e.g., maternal medical illness, obstetric complications, etc.), psychosocial risk factors (e.g., no prenatal care), and so one. Still in other implementations, training component 108 can train separate models tailored to different types of cardiotocograph monitoring devices (e.g., internal verses external, different device makes/models, etc.).

The type of machine learning model architecture employed for cardiotocograph pattern identification model 112 can vary. For example, cardiotocograph pattern identification model 112 can employ various types of machine algorithms, including (but not limited to): deep learning models, neural network models, deep neural network models (DNNs), convolutional neural network models (CNNs), generative adversarial neural network models (GANs), LSTM models, attention-based models, transformers, or a combination thereof. In some embodiments, cardiotocograph pattern identification model 112 can additionally or alternatively employ a statistical-based model, a structural based model, a template matching model, a fuzzy model or a hybrid, a nearest neighbor model, a naïve Bayes model, a decision tree model, a linear regression model, a k-means clustering model, an association rules model, a q-learning model, a temporal difference model, or a combination thereof.

Regardless of the specific type of machine learning model architecture employed, training component 108 can employ a data-driven supervised machine learning process to train and develop one or more cardiotocograph pattern identification models 112. In a supervised learning framework, the learning system is first given examples of data by which human experts or annotators apply classification labels to a corpus of data. The class labels are then employed by the learning algorithm to adapt and change its internal, mathematical representation (such as the behavior of artificial neural networks) of the data and mapping to some predication of classification etc. The training consists of iterative methods employing numerical, optimization techniques that reduce the error between the desired class label and the algorithm's prediction. The newly trained model is then given new data as an input and, if trained well, can classify or otherwise provide assessment of novel data.

In this regard, the training data employed to train the one or more cardiotocograph pattern identification models 112 can comprise at least some labeled cardiotocograph data annotated with information identifying known patterns and their corresponding physiological events. In the embodiment shown, this training data is represented as cardiotocograph training data 102. Cardiotocograph training data 102 can include previously recorded cardiotocograph data captured for a plurality of different mothers and their respective unborn fetuses over a duration of time. In an embodiment, the duration of time can include the duration of labor, such as from the onset of labor to delivery, or a portion of time there between. Additionally, or alternatively, cardiotocograph training data 102 can include cardiotocograph data recorded for subjects (e.g., respective mothers and their unborn fetuses) at any point during their pregnancy, such as that recorded in association with a monitoring/check-up session.

In one or more embodiments, cardiotocograph training data 102 for each subject (e.g., each mother and fetus combination) can comprise both FHR tracing data and UA tracing data captured simultaneously, referred to herein as FHR-UA tracing data. The FHR tracing data can include information that can track the FHR over time, while the UA tracing data can include information that can track the UA over time. The frequency of sampling of the FHR measurements and the UA measurements can vary depending on the type of cardiotocograph device used. For example, the sampling frequency can be every millisecond, every second, every few seconds, every 10 seconds, and so on. In accordance with conventional FHR monitoring systems, the FHR and UA recordings can typically be in the form of graphical tracings that can cither be printed on a continuous strip of paper and/or be displayed on a graphical display monitor. The simultaneous recordings of the FHR and the UA can be typically performed by two separate transducers, one for the measurement of the FHR and a second one for the UA. The transducers can be either external or internal.

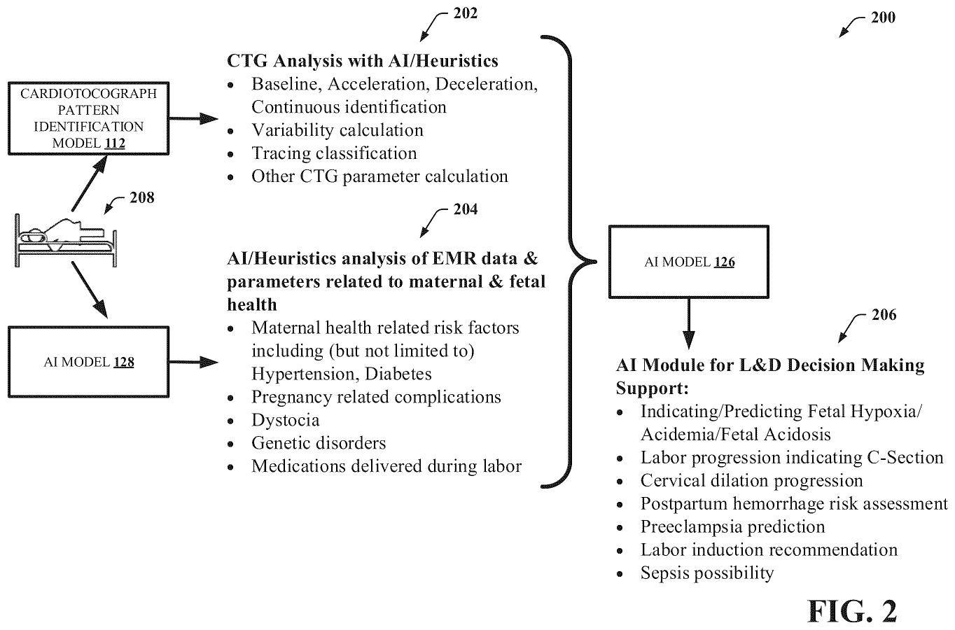

FIG. 2 illustrates a flow diagram of an example, non-limiting method 200 that can generate labor and delivery predictions by analyzing CTG analysis data and maternal health analysis data in accordance with one or more embodiments of the disclosed subject matter. Repetitive description of like elements and/or processes employed in respective embodiments is omitted for sake of brevity.

With continued reference to FIG. 1, in an embodiment, AI model 126 can generate data 206 comprising one or more labor and delivery predictions applicable to one or more fetuses and a mother of the one or more fetuses, during labor of the mother, by analyzing data 202 comprising CTG analysis data of the one or more fetuses and the mother generated by AI model 128 and data 204 comprising maternal health analysis data of the mother generated by cardiotocograph pattern identification model 112. For example, hypertension combined with other clinical factors can be good indicators of acidosis during labor, and AI model 126 can analyze multiple clinical factors to confirm such a hypothesis and generate a prediction with a confidence interval based on the hypothesis.

In this regard, data 202 can comprise one or more features selected from a group consisting of an FHR baseline value calculation, FHR acceleration, deceleration, and contraction event identification and calculation of related parameters, variability in FHR value calculation generated via rules and AI algorithms, fetal tracing classification generated via a rule-based calculation to classify tracing, and one or more additional CTG analysis data types. Cardiotocograph pattern identification model 112 can generate data 202 by processing FHR data of the one or more fetuses and UA data of the mother. Similarly, data 204 can comprise one or more features selected from a group consisting of maternal health related risk factors including, but not limited to hypertension and diabetes, pregnancy related complications, dystocia, genetic disorders and one or more additional maternal health analysis data types. AI model 128 can be an AI model for maternal health analysis that can generate data 204 by employing AI, rule-based algorithms and heuristics algorithms to analyze EMRs and health parameters of the mother such as, for example, maternal health indicators, HR, medical history, recent medical history, genetic profile, medications administered during labor, etc. For example, AI model 128 can analyze an HR of the mother and generate insights on anomalies detected in the mother's HR. AI model 126 can ingest data 202 and data 204 to generate data 206. Data 206 can comprise one or more features selected from a group comprising indications or predictions of fetal hypoxia, fetal acidemia, fetal acidosis, labor progression indicating a C-section, cervical dilation progression, a postpartum hemorrhage risk assessment, a preeclampsia prediction, a labor induction recommendation, a sepsis possibility, and one or more additional labor and delivery predictions.

In an embodiment, AI model 126 can be accessible to entities (e.g., hardware, software, AI, neural networks, machine and/or user) as part of an application for clinical decision support at a user interface (UI) of computing device 106. For example, an application for clinical decision support can be provided to a clinician, and the application can be supported by clinical insights generated by AI model 126, where AI model 126 can analyze the clinical insights generated by AI model 128 and cardiotocograph pattern identification model 11, and where the clinical insights generated by AI model 126 can be employed by the clinician to execute relevant actions. As such, one or more embodiments of the present disclosure can employ a combination of AI and heuristics to perform calculations and generate labor and delivery predictions that can provide an overview of the health of a mother and the one or more fetuses of the mother during labor and through delivery of the fetuses.

In an embodiment, cardiotocograph pattern identification model 112 and AI model 128 can perform a first level of processing wherein cardiotocograph pattern identification model 112 and AI model 128 can ingest numerical data, time series data and/or waveform data and perform AI-based and heuristics-based analyses on the ingested data to respectively generate data 202 and data 204. Thereafter, AI model 126 can perform a second level of processing wherein AI model 126 can ingest data 202 and data 204 to generate data 206. In an embodiment, the data ingested by cardiotocograph pattern identification model 112 and AI model 128 can be generated by devices employed to perform fetal monitoring and maternal monitoring, as illustrated at 208, during labor of a pregnant woman and from EMRs that can be generated prior to and/or during the labor. In this regard, the various embodiments disclosed herein can present an AI-powered system for labor and delivery that can provide descriptive, predictive, and/or prescriptive clinically relevant analyses related to labor and delivery. Further, the various embodiments disclosed herein can act as an AI powered entity/nurse/expert to assist caregivers in decision making.

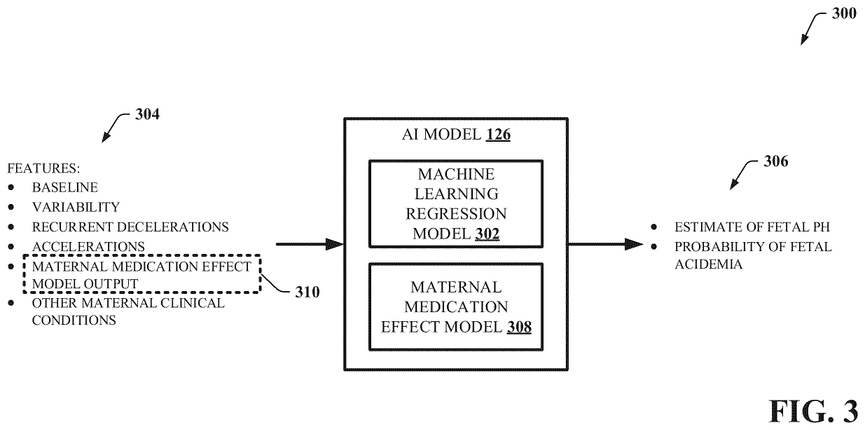

For example, during labor, clinically relevant information related to the mother and the one or more fetuses can be generated via one or more devices employed in various embodiments for fetal monitoring and maternal monitoring. The one or more devices referenced herein can comprise an FMS, a CTG, internal or external leads, sensors, probes, etc., and the information captured by the one or more devices can include values computed by the one or more devices, for example, in the patient room of a hospital, clinic or other medical facility during the labor. In addition to data from the one or more devices (e.g., FMS, CTG, etc.), EMR information and parameters captured via laboratory tests, check-ups by clinicians, notes generated by nurses or other entities (e.g., hardware, software, AI, neural network, machine, and/or user), and so on, can be input to computing device 106. Such data can be accessed by cardiotocograph pattern identification model 112 and AI model 128 to respectively generate data 202 and data 204. In an embodiment, AI model 126 can be a multistage AI model comprising respective models directed to predicting respective ones of the one or more labor and delivery predictions. An example of the same is illustrated in FIG. 3.

FIG. 3 illustrates a flow diagram of an example, non-limiting method 300 that can generate an estimate of fetal pH and predict the probability of fetal acidemia by analyzing CTG analysis data and maternal health analysis data in accordance with one or more embodiments of the disclosed subject matter. Repetitive description of like elements and/or processes employed in respective embodiments is omitted for sake of brevity.

In an embodiment, AI model 126 can comprise an LSTM model, a machine learning regression model, and/or one or more additional models, wherein respective models comprised in AI model 126 can be directed to generating respective ones of the one or more labor and delivery predictions comprised in data 206. In this regard, AI model 126 can also employ standard machine learning methods such as, for example, logistic regression, SVMs, decision trees, etc. depending on the respective labor and delivery predictions. For example, some predictions generated by AI model 126 can include binary outcomes and a logistic regression framework can be suitable to generate such binary outcomes. Some examples of binary outcomes can include a prediction for whether a C-section is needed in a particular labor scenario, hypoxia predictions, etc. On the contrary, other predictions generated by AI model 126 can be multiclass predictions as opposed to binary predictions, and AI model 126 can employ suitable machine learning techniques to generate such predictions.

For example, data 304 can comprise features from data 202 and data 204 that AI model 126 can employ to estimate data 306. For example, AI model 126 can employ baseline, variability, recurrent FHR decelerations, maternal medication effect model output, FHR accelerations and other maternal clinical conditions to estimate an intrapartum fetal pH value and the probability of fetal acidemia for a fetus during labor of a mother of the fetus. To generate data 306, AI model 126 can select machine learning regression model 302 from the models comprised in AI model 126, and AI model 126 can select machine learning regression model 302 based on the type of data to be processed and the prediction to be generated, for example, to estimate the intrapartum fetal pH value and the probability of fetal acidemia for the fetus.

In addition to models that can generate labor and delivery predictions such as the predictions comprised in data 206, AI model 126 can comprise models that can process certain features from data 202 and data 204 to generate additional features of data 304. For example, baseline and variability of FHR can be significant CTG analysis parameters for analyzing tracings, and the baseline and variability can be indicative of certain medical conditions of a fetus. However, a low baseline FHR and low variability of the FHR can also be a side effect of medications administered to a mother during labor, as opposed to parameters resulting from a medical condition of a fetus. In an embodiment, AI model 126 can comprise maternal medication effect model 308 that can be employed by AI model 126 to trace the degree of effect that medications administered to the mother can have on the CTG analysis parameters such as baseline, variability, etc. Maternal medication effect model 308 can be a statistical model that can be trained (e.g., by training component 108), based on retrospective data previously recorded over the course of multiple deliveries of a plurality of mothers, to account for the effect of medications or drugs administered to the mother during labor on the CTG analysis parameters.

For example, the retrospective data can associate each drug with the range of percentage decrease that can be caused in the FHR baseline and FHR variability, and maternal medication effect model 308 can be trained to perform statistical modeling based on such data. An example of such retrospective data is illustrated in Table 1. Thus, AI model 128 can capture information about medications delivered during labor in data 204 and maternal medication effect model 308 can perform statistical modeling to identify the effects of the medications on the FHR and FHR variability. The information that can be generated by maternal medication effect model 308 is listed as maternal medication effect model output, at 310, in data 304 and can be employed by AI model 126 to analyze other conditions related to the mother and the one or more fetuses. For example, maternal medication effect model output can be employed by machine learning regression model 302 in conjunction with CTG analysis features and maternal health analysis features comprised in data 304 to estimate fetal pH for demise, hypoxia or acidosis conditions. The fetal pH estimated by machine learning regression model 302 for a fetus can be an estimate of a value that is typically determined in a laboratory from a pH value measured from the umbilical cord or a baby after the baby is delivered. The rise and fall of the estimated fetal pH value can be monitored over time by AI model 126 to determine fetal hypoxia, fetal acidemia and/or fetal acidosis conditions by comparing the estimated fetal pH value to known fetal pH values associated with various medical conditions of the fetus. For example, machine learning regression model 302 can be employed to categorize a fetal strip, which is a chart that can monitor FHR and UA for one or more fetuses and the mother of the one or more fetuses, to determine fetal hypoxia.

Machine learning regression model 302 can be one type of regression model employed by AI model 126, and AI model 126 can employ other regression models to generate different labor and delivery predictions since there can be multiple approaches to regression such as, for example, linear regression or other types of regression. Generally, regression models can be employed to model continuous parameters such as a fetal pH value. In some embodiments, AI model 126 can also employ classification models to classify data. Thus, AI model 126 can comprise different models directed to various types of labor and delivery related estimations or predictions.

TABLE 1
Data from National Institute of Child Health and Human Development
(NICHD), a clinical standard for CTG analysis

| Medication | Change in FHR |
|---|---|
| Narcotics | Decrease in variability, decrease in frequency of |
| | accelerations |
| Butorphanol | Transient sinusoidal FHR pattern, slight increase |
| | in baseline rate |
| Cocaine | Decrease in FHR variability |
| Corticosteroids | Decrease in FHR variability with betamethasone, |
| | but not |
| Magnesium sulfate | Decrease in FHR variability, clinical insignificant |
| | decrease in |
| Terbutaline | Increase in baseline rate |
| Zidovidine | No change |

In an embodiment, machine learning regression model 302 can be trained by training component 108 based on fetal pH data previously recorded for a plurality of mothers and their respective fetuses. For example, the fetal pH data can be obtained from post-delivery medical notes generated for the plurality of mothers and stored in memory 122 as part of the training data to train AI model 126. The post-delivery medical notes can comprise pH data obtained through fetal scalp blood or through umbilical cord blood and documented in the post-delivery medical notes. Training component 108 can employ the estimated fetal pH data as output data and relevant features selected from data 202 and data 204 as input data to train machine learning regression model 302 to recognize patterns in specific input data ingested by AI model 126 and generate labor and delivery predictions corresponding to the specific input data. During training, training component 108 can manipulate machine learning regression model 302 to the specific data to be processed to generate respective labor and delivery predictions of data 206.

In another example, AI model 126 can employ an LSTM model to generate a different prediction. For example, AI model 126 can be employed to predict labor progression trends to determine whether a delivery can be a C-section or a normal vaginal birth, and predicting whether a C-section is needed can involve a different type of modelling than that of a regression model. The LSTM model can be trained (e.g., by training component 108) based on retrospective or historical data from previous births to predict a future trend based on past trends. For example, the LSTM can be trained on clinical trends recorded over 7 hours. The 7 hours of data can be split into a first section comprising 4 hours of data and a second section comprising 3 hours of data, and the LSTM model can be trained to predict clinical trends recorded during the second section based on the clinical trends recorded during the first section. Training component 108 can continuously feed such trends to the LSTM model to train the LSTM model, and the LSTM model can learn the behaviors that can be employed to predict certain labor and delivery trends such as, for example, cervical dilation, based on new data associated with a mother undergoing labor. Thus, multiple different models can be trained to predict different labor and delivery features. Training component 108 can employ data labelled by clinical experts to train the different models to predict certain patterns.

In an embodiment, pre-processing component 110 can perform pre-processing of data to generate training data that can be employed by training component 108 to train the different models of AI model 126. For example, pre-processing component 110 can perform data cleaning to extract valuable data from information comprised in a tabular format, and pre-processing component 110 can further transform the data and structure the data in a format that can be employed to train a model. In another example, pre-processing component 110 can collate data generated by different entities (e.g., hardware, software, AI, neural network, machine and/or user (e.g., nurses, clinicians, physicians, etc.)) prior to the data being employed by training component 108 to train a model. Doing so can prevent an ongoing workflow for a patient from being interrupted. For example, during labor, an entity can hourly enter information into an application that can interact with embodiments of the present disclosure, and pre-processing component 110 can extract valuable data from the information entered to the application and transform the extracted data into trends that can be employed to train different models of AI model 126. Pre-processing component 110 can also assist with data acquisition in case of data generated by devices such as FMSs or CTGs that can be in direct contact with the mother or in case of data from EMRs.

In an embodiment, in addition to identifying and collating valuable data, pre-processing component 110 can perform other types of processing. For example, pre-processing component 110 can perform data prioritization. For example, a blood pressure value noted by a physician can be more accurate as opposed to a blood pressure value inflated by a cough, or an HR from an electrocardiogram (EKG) lead can be more accurate than an HR detected by an SpO2 lead, and pre-processing component 110 can select values based on accuracy to ensure that AI model 126 can be trained on the most accurate data available. In an embodiment, pre-processing component 110 can also scale data to normalize the range of data, for example, to fit data between the values of zero (0) and one (1) for models that can only accept a normalized data range. In general, pre-processing component 110 can perform data acquisition, data cleaning, data normalization, data prioritization, etc. to generate training data that can be employed by training component 108 to train AI model 126 and the plurality of models comprised in AI model 126.

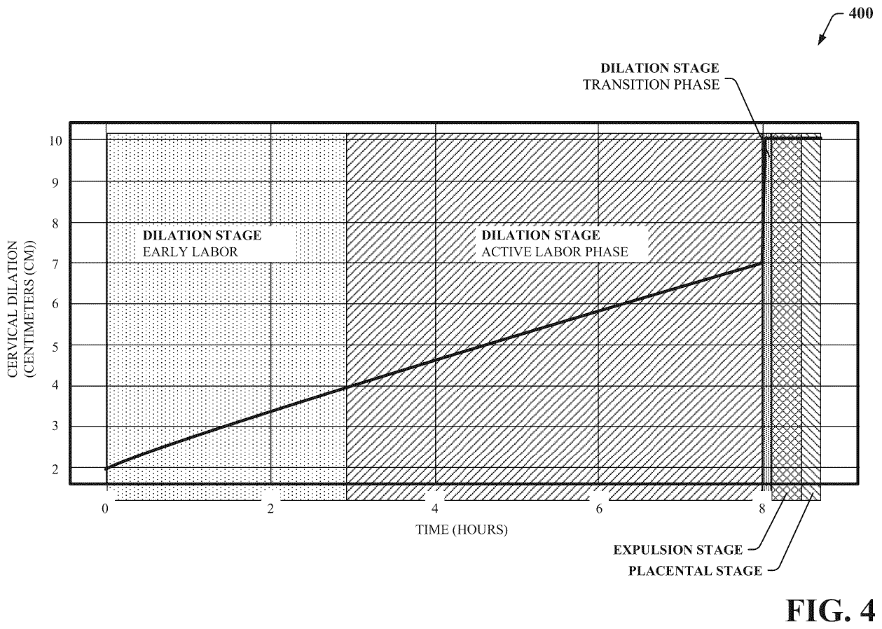

FIG. 4 illustrates an example, non-limiting graph 400 of a labor progression curve in accordance with one or more embodiments of the disclosed subject matter. Repetitive description of like elements and/or processes employed in respective embodiments is omitted for sake of brevity.

As discussed with reference to FIG. 3, in some embodiments, AI model 126 can employ an LSTM model to generate a labor and delivery prediction associated with a mother in labor. For example, AI model 126 can be employed to predict labor progression trends to determine whether a delivery can be a C-section or a normal vaginal birth, and predicting whether a C-section is needed. Labor progression can refer to the progression of cervical dilation and can be commonly plotted via a Dawse-Redman curve, a partogram or other labor progression curve. Non-limiting graph 400 illustrates an exemplary labor progression view or curve for a normal/vaginal delivery of a baby. Non-limiting graph 400 maps cervical dilation in centimeters (cm) versus time in hours to depict how the cervix can be expected to dilate or how cervical dilation can be expected to progress with time during a normal delivery of a baby. For example, the cervix needs to be at a certain diameter for the baby to be delivered and the dilation of the cervix needs to occur in a certain manner from the early stages of labor to the later stages of labor. In this regard, non-limiting graph 400 illustrates the various dilation stages, the expulsion stage and the placental stage. Thus, if a labor progression curve for a mother can be closely aligned with the cervical dilation trend illustrated in non-limiting graph 400, the mother can be expected to have a normal delivery. However, the probability of a C-section can increase if the labor progression curve for the mother significantly deviates from the cervical dilation trend illustrated in non-limiting graph 400. For example, if the cervical dilation is not enough for a vaginal delivery of a baby, a C-section can be performed.

In actuality, a cervical dilation trend can alter over time based on different parameters such as, for example, the number of children that the mother has had in the past. For example, a mother of two children can labor faster when delivering a third child due to faster cervical dilation. Epidural drugs such as Pitocin® that can be administered to induce labor in the mother or hormones can also alter the cervical dilation trend over time. As a result, timely intervention by entities (e.g., hardware, software, AI, neural network, machine and/or users) can be employed to maintain the labor progression curve close to the labor progression curve illustrated in non-limiting graph 400. For example, nurses or clinicians can employ different techniques or medications to maintain the labor progression curve for a mother, close to the standard normal transition illustrated in non-limiting graph 400. Embodiments of the present disclosure can assist various entities by identifying instances when such intervention is needed. As such the time spent in manual decision making can be significantly reduced by employing AI-based techniques as opposed to visual or manual analysis. In an embodiment, the cervical dilation for a mother during labor can be documented over time and entered to computing device 106 by an entity (e.g., hardware, software, AI, neural network, machine and/or user), and the information can be accessed by AI model 128 as a maternal parameter to generate maternal health analysis factors (e.g., data 204). The maternal health analysis factors can be employed by AI model 126 to identify the labor progression for the mother and determine whether a C-section is needed. In this regard, output component 132 can output data generated by AI model 126 that can be indicative of a C-section, to the UI of a computing device such as computing device 106.

In an embodiment, the data output by output component 132 can be in a format different from a graphical format, and the entity can select a button presented on the UI, via a mouse or other selection device, to generate a graph illustrating a labor progression curve based on the data output by output component 132. The graph can be generated to visualize a comparison between a labor progression of a mother and the labor progression of a normal delivery such as illustrated by non-limiting graph 400, and the entity can analyze the cervical dilation trend presented by the graph. In another embodiment, the entity can evaluate the data output by output component 132 to determine whether a C-section is needed. In yet another embodiment, output component 132 can output a prediction with a confidence interval generated by AI model 126, which confident interval can indicate the probability of a C-section for the mother. AI model 126 can generate data in various formats such as, for example, JavaScript Object Notation (JSON), comma separated values (CSV), etc. Thus, AI model 126 can analyze data that can contribute to a cervical dilation trend and generate an output that can indicate whether the cervical dilation trend for a mother is significantly deviating from that observed for a vaginal birth. In an embodiment, alert component 130 can alert the entity if the cervical dilation trend for the mother varies outside of a threshold range.

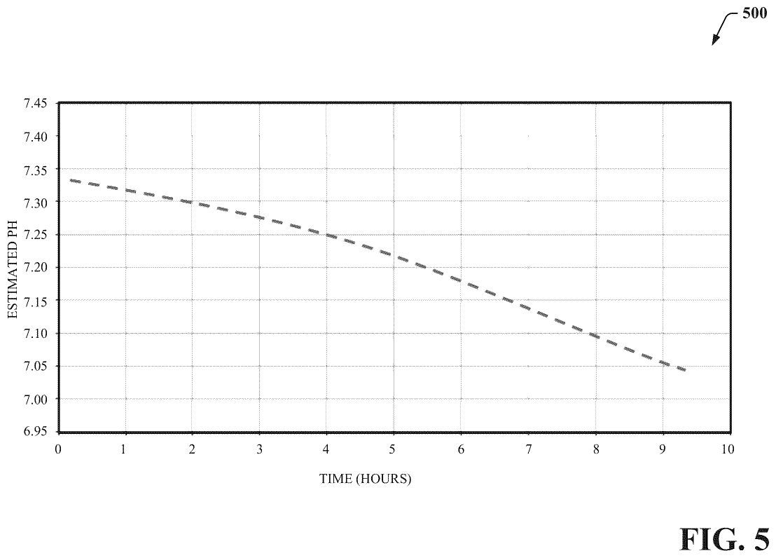

In an embodiment, analysis of fetal pH can also contribute to predicting the need for a C-section delivery. This is further described with reference to FIG. 5.

FIG. 5 illustrates an example, non-limiting graph 500 of a fetal pH curve in accordance with one or more embodiments of the disclosed subject matter. Repetitive description of like elements and/or processes employed in respective embodiments is omitted for sake of brevity.

A fetal pH curve such as that illustrated by non-limiting graph 500 can be indicative of a C-section. For example, a declining fetal pH over time can indicate a slow cervical dilation and thereby, a need to perform a C-section to prevent fetal acidosis from beginning or to prevent the blood pH value from dropping below a threshold value. In an embodiment, AI model 126 can predict a fetal pH trend over time, based on an existing fetal pH trend for a fetus. For example, with continued reference to FIG. 3, AI model 126 can employ machine learning regression model 302 to estimate fetal pH values over time from data 304, and AI model 126 can analyze the estimated fetal pH values over a defined time interval to predict the fetal pH values that can be expected for the fetus.

In an embodiment, the fetal pH values predicted by AI model 126 can be visualized as a graph to represent the medical condition of a fetus. For example, output component 132 can output the fetal pH values predicted by AI model 126 as numerical data to the UI of computing device 106, and an entity (e.g., hardware, software, AI, neural network, machine and/or user) can select a button presented on the UI, via a mouse or other selection device, to generate a graph illustrating a fetal pH curve, similar to that illustrated by non-limiting graph 500. In conjunction with cervical dilation trends predicted by AI model 126, the fetal pH curve can be employed by the entity to analyze whether the mother can have a vaginal delivery or a C-section. In another embodiment, output component 132 can output a confidence interval generated by AI model 126, which confidence interval can be indicative of whether a C-section is needed for the mother.

In an embodiment, output component 132 can display cervical dilation trends and fetal pH trends as continuous, time varying graphs based on the predictions made by AI model 126. Based on such outputs, an entity (e.g., hardware, software, AI, neural network, machine and/or user) can intervene to prevent the cervical dilation curve and/or the fetal pH curve associated with a mother from deviating outside of acceptable bounds indicative of a healthy delivery. AI model 126 can generate data in various formats such as, for example, JSON, CSV, etc. In some embodiments, AI model 126 can be accessed by the entity to generate specific labor and delivery predictions at the click of a button on the UI. In other embodiments, AI model 126 can automatically generate specific labor and delivery predictions based on data ingested by AI model 126.

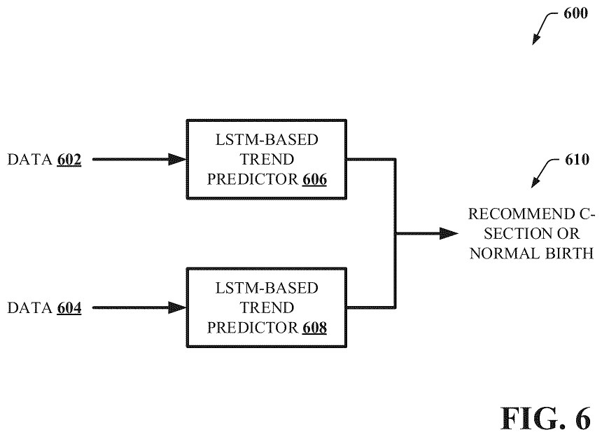

FIG. 6 illustrates a flow diagram of an example, non-limiting method 600 that can generate a recommendation for a C-section in accordance with one or more embodiments of the disclosed subject matter. Repetitive description of like elements and/or processes employed in respective embodiments is omitted for sake of brevity.

With continued reference to FIGS. 3-5, non-limiting method 600 illustrates LSTM models that can be employed by AI model 126 to predict, during a period of labor, whether a pregnant mother can have a normal/vaginal birth or a C-section. For example, LSTM-based trend predictor 606 can be an LSTM model employed by AI model 126 to generate labor progression and cervical dilation predictions based on data 602. Similarly, LSTM-based trend predictor 608 can be an LSTM model employed by AI model 126 to generate fetal pH predictions. As discussed elsewhere herein, AI model 126 can be a multistage AI model comprising one or more models. As such, AI model 126 can comprise LSTM-based trend predictor 606 and LSTM-based trend predictor 608.

In an embodiment, LSTM-based trend predictor 606 and LSTM-based trend predictor 608 can be trained (e.g., by training component 108) based on retrospective or historical data from previous births to predict future trends based on past trends. For example, LSTM-based trend predictor 606 can be trained on cervical dilation/labor progression trends recorded over time. In this regard, data 602 can comprise cervical dilation data that can be employed to generate a labor progression curve such as that illustrated in non-limiting graph 400. Data 602 can comprise cervical dilation data previously recorded for a plurality of mothers that can be employed to train LSTM-based trend predictor 606. Similarly, LSTM-based trend predictor 608 can be trained on fetal pH trends recorded over time. In this regard, data 604 can comprise fetal pH values that can be employed to generate a fetal pH curve such as that illustrated in non-limiting graph 500. Data 604 can comprise fetal pH data previously recorded for a plurality of mothers that can be employed to train LSTM-based trend predictor 608.

Data 602 and data 604 can respectively comprise time series data that can be split into first and second time periods, and LSTM-based trend predictor 606 and LSTM-based trend predictor 608 can be respectively trained (e.g., by training component 108) to predict clinical trends occurring during the second time periods based on clinical trends observed during corresponding first time periods. For example, 7 hours of cervical dilation data can be split into a first section comprising the first 4 hours of data and a second section comprising the last 3 hours of data, and LSTM-based trend predictor 606 can be trained to predict cervical dilation and labor progression trends recorded during the second section based on the cervical dilation and labor progression trends recorded during the first section. LSTM-based trend predictor 608 can be similarly trained.

Data 602 and data 604 can comprise large amounts and numerous hours of data, and training component 108 can continuously feed such data to LSTM-based trend predictor 606 and LSTM-based trend predictor 608 to train the LSTM models. Based on the training, LSTM-based trend predictor 606 and LSTM-based trend predictor 608 can learn the behaviors that can be employed to predict respective labor and delivery trends based on new data associated with a mother undergoing labor. Data 602 and data 604 can comprise labelled data that can be employed to train LSTM-based trend predictor 606 and LSTM-based trend predictor 608. As discussed elsewhere herein, based on the training, the clinical trends predicted by LSTM-based trend predictor 606 and LSTM-based trend predictor 608 can be employed by AI model 126 to recommend a C-section or a normal birth for a fetus, as illustrated at 610.

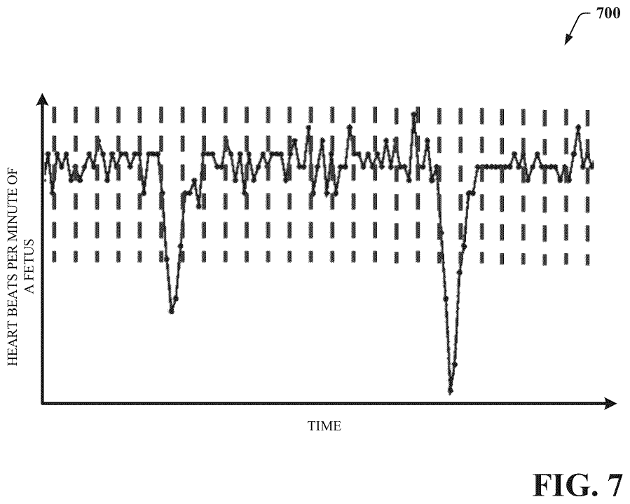

FIG. 7 illustrates an example, non-limiting graph 700 based of FHR variability in accordance with one or more embodiments of the disclosed subject matter. Repetitive description of like elements and/or processes employed in respective embodiments is omitted for sake of brevity.

As discussed with reference to at least FIG. 1, cardiotocograph pattern identification model 112 can generate CTG analysis data (e.g., data 202 in FIG. 2) associated with one or more fetuses of a mother in labor by processing FHR data of the one or more fetuses and UA data of the mother. For example, cardiotocograph pattern identification model 112 can perform CTG analysis of waveform data comprising FHR data of the one or more fetuses and UA data of the mother to generate the CTG analysis data. The CTG analysis data can comprise features such as an FHR baseline value calculation, FHR acceleration, deceleration, and contraction event identification and calculation of related parameters, FHR variability calculation generated via rules and AI algorithms, fetal tracing classification generated via a rule-based calculation to classify tracing, and one or more additional CTG analysis data types.

In this regard, non-limiting graph 700 illustrates a graph of FHR variability versus time for a fetus. Non-limiting graph 700 can be representative of the values from Table 2. A method employed by cardiotocograph pattern identification model 112 to calculate the FHR variability from waveform data is described hereinafter. The waveform data associated with the values in Table 2 can be pre-processed by pre-processing component 110 to break down a waveform segment of X minutes into N segments of Y seconds each, where X, Y, and N represent positive numeric values. In an embodiment, Y can be selected empirically according to the equation, N x Y=X. Thereafter, cardiotocograph pattern identification model 112 can employ an algorithmic/heuristic method to compute an absolute difference in maxima and minima of each segment, D, wherein D can represent an array of P different values of such differences. Cardiotocograph pattern identification model 112 can identify the median of array D=M, wherein M can represent the variability value of the segment of waveform

TABLE 2
Data to compute FHR variability.
Example

| Total waveform duration (X) | 5 minutes |
|---|---|
| Number of segments (N) | 10 |
| Each segment's duration (Y) | 30 seconds |
| Array D (D) | 10, 15, 60, 20, 20, |
| | 20, 90, 10, 20 |
| Variability (M) = median of D | 20 |

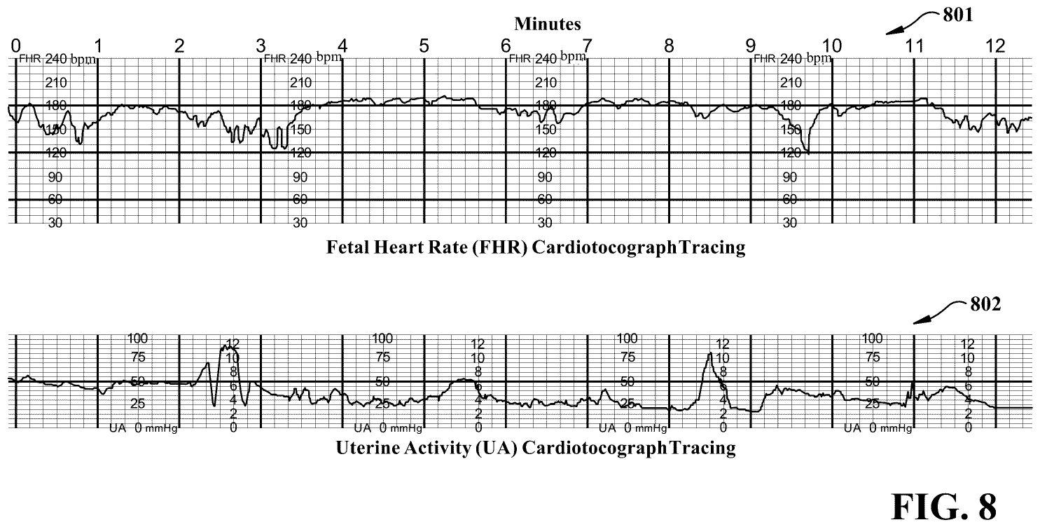

FIG. 8 presents example FHR and UA tracings in accordance with one or more embodiments of the disclosed subject matter. FHR tracing 801 corresponds to an FHR tracing for an unborn fetus and UA tracing 802 corresponds to a simultaneously recorded UA tracing for the mother of the unborn fetus. The FHR tracing 801 plots the HR of the fetus over time while the UA tracing plots the mother's UA over time (e.g., measure in minutes).

CTG is widely employed in pregnancy as a method of assessing fetal well-being, particularly during labor and delivery. CTG records changes in the FHR and its temporal relationship to uterine contractions. In CTG, simultaneous recordings are performed by two separate transducers, one for the measurement of the FHR and a second one for the UA. The transducers can be either external or internal. The cardiograph recordings are typically in the form of graphical tracings that are either printed on a continuous strip of paper and/or displayed on a graphical display monitor. FHR and UA tracings such as those depicted in FIG. 8 can typically be visually analyzed, manually by an obstetric medical team of nurses, resident physicians, nurse midwives and attending physicians over the course of labor to identify abnormal patterns. These patterns can include patterns in the FHR tracing alone, the UA tracing alone, as well as combined patterns in the simultaneous recordings. The identification of the patterns can help in assessing various parameters and/or conditions associated with the fetus and/or the mother, such as baseline FHR, contraction duration and frequency, contraction nature (e.g., late, early, variably), FHR variability, and presence of tachysystole. Assessment of these parameters/conditions can play a vital role in identifying fetal abnormalities and the need for intervention during labor.

For example, obstetric clinicians are trained to visually examine the FHR-UA tracings to identify distinct patterns in the graphical representations that have been known to correspond to defined physiological events associated with the fetus and/or the mother. Guidelines defining the distinct patterns and the physiological events/conditions associated with them have been defined in medical literature. For example, individual uterine contractions are seen as peaks on the UA tracing 802. The guidelines for evaluating contraction patterns from a UA tracing such as FHR tracing 801 generally involve identifying the frequency of the peaks/contractions (e.g., number of contractions per defined time interval), the duration of the contractions, and the intensity of the contractions. In another example, FHR accelerations are seen as abrupt peaks on the FHR tracing 801 while FHR decelerations are seen as abrupt valleys or spikes. The guidelines for evaluating FHR tracing data are generally based on identifying and evaluating the degree of variability in the graphical representation (e.g., number of peaks and valleys, frequency of peaks and valleys, amplitude of the peaks and valleys, etc.), identifying and evaluating peaks and valleys corresponding to accelerations and decelerations, and correlating the peaks and valleys in the FHR tracing with contraction peaks. Trained clinicians can extract these parameters from the UA tracing visually by examining the patterns in the graphical representation of the data.

With reference to FIGS. 1 and 8, in an embodiment, the disclosed techniques can automate this visual/manual abnormal pattern identification process by training one or more cardiotocograph pattern identification models 112 to identify the abnormal patterns and/or other defined patterns in the cardiotocograph data that correspond to defined physiological events associated with the fetus and/or the mother. For example, the defined physiological events can include, but are not limited to, an FHR acceleration, an FHR deceleration, a period of FHR variability, and a uterine contraction. Various other physiological events associated with the fetus and/or mother that can also be correlated to distinct patterns in the cardiotocograph data are also envisioned. For example, some other physiological events that can be represented by defined patterns in the FHR-UA data can include but are not limited to: fetal tachycardia, fetal bradycardia, saltatory variability, variable decelerations, late decelerations, persistent late decelerations with loss of beat-to-beat variability, non-reassuring variable decelerations associated with loss of beat-to-beat variability, prolonged severe bradycardia, and sinusoidal pattern. The number of different patterns and correlated physiological events can vary.

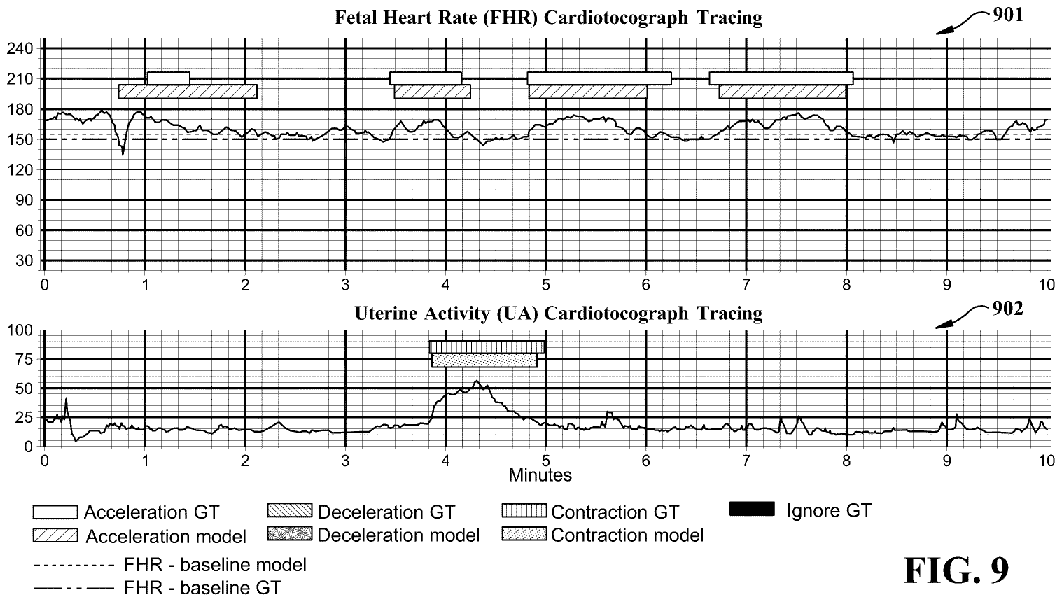

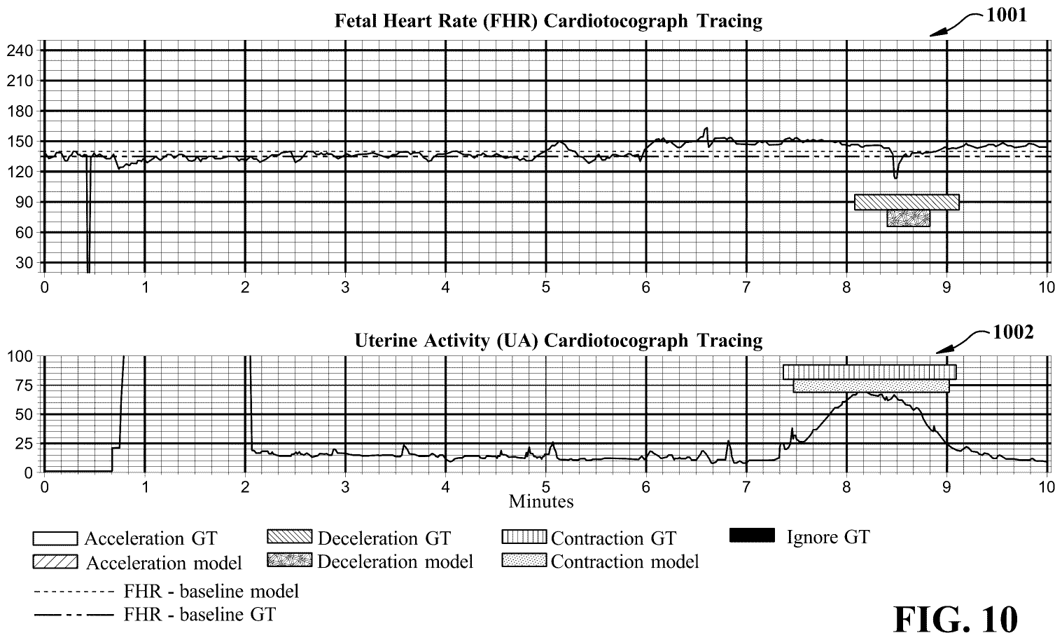

As noted above, to facilitate this end, training component 108 can train the cardiotocograph pattern identification models 112 to identify the defined patterns in cardiotocograph data corresponding to the defined physiological events by employing supervised learning processes that involve training cardiotocograph pattern identification models 112 to learn from labeled cardiotocograph training data 102. In this regard, at least some of cardiotocograph training data 102 can comprise annotation information applied thereto (or otherwise associated therewith) that identify one or more patterns in the cardiograph data (e.g., FHR-UA tracing data) and their corresponding physiological events or conditions. The number of different patterns and correlated physiological events can vary. In some embodiments, the annotated cardiotocograph data can include marked-up FHR-UA tracings with annotation marks applied directly to the graphical tracings, as illustrated in FIGS. 9 and 10.

In this regard, FIGS. 9 and 10 present example FHR and UA tracings comparing the model identified patterns with the ground truth annotated patterns in accordance with one or more embodiments of the disclosed subject matter. Tracings 901 and 1001 correspond to FHR tracings and tracings 902 and 1002 correspond to the UA tracings. In accordance with these examples, each set of tracings (e.g., tracings 901 and 902 corresponding to one set and tracings 1001 and 1002 corresponding to another set) represent a 10-minute window of time. As described in greater detail below, in some embodiments, the cardiotocograph pattern identification model 112 can be trained to identify patterns in input data samples that consist of FHR-UA tracing data tracked for a defined window of time, such as 10 minutes or another timeframe (e.g., each input sample can represent a 10-minute window). The respective tracings are marked with graphical markings in the form of horizontal bars over (or otherwise in alignment with) the distinct patterns in the tracing data that correspond to defined physiological events. In these examples, the physiological events include an FHR acceleration, an FHR deceleration and a contraction. In both FIGS. 9 and 10, the upper bars with solid fill (e.g., without the slanted lines) correspond to the manually annotated ground truth, while the lower bars with the slanted lines correspond to the patterns automatically identified by a trained version of cardiotocograph pattern identification model 112. The manually annotated ground truth can also include manually identified regions/patterns in the cardiotocograph data that an expert understands as not corresponding to any defined physiological event or condition yet can assume could potentially be misinterpreted by the model during training (e.g., due to a tendency for manual reviewers to misinterpret these types of patterns). By annotating these “ignore” regions, the accuracy of the model can be further enhanced to minimize false positive detections.

With reference again to FIG. 1, in an embodiment, the format of cardiotocograph training data 102, including the annotated cardiotocograph data (and any unannotated samples if included), can vary. In some embodiments, the format of cardiotocograph training data 102 can include digital graphical FHR-UA tracings that can correspond to those depicted in FIGS. 8-10. With these embodiments, the digital graphical FHR-UA tracings can be annotated with manually applied mark-up data in the manner shown in FIGS. 8-10 and/or in a similar manner. Additionally, or alternatively, cardiotocograph training data 102 can include the raw signal or waveform data used to generate the digital graphical FHR-UA tracings. For example, the raw signal data for the FHR information can include FHR measurements recorded as a function of time with corresponding time stamps for each measurement. Likewise, the raw signal data for the UA information can include UA measurements recorded as a function of time with corresponding time stamps for each measurement.

In an embodiment, the format of cardiotocograph data supplied as input to cardiotocograph pattern identification model 112 (e.g., during inferencing and/or training) can be the same as that of cardiotocograph training data 102 or optionally transformed into a different machine-readable format by pre-processing component 110, as described elsewhere herein. For example, in some embodiments in which cardiotocograph training data 102 can be received as a digital graphical FHR-UA tracing, pre-processing component 110 can convert the digital graphical FHR-UA tracing data into its corresponding raw signal data prior to input into cardiotocograph pattern identification model 112. Pre-processing component 110 can further translate the manually applied ground truth annotation data from the annotated digital graphical FHR-UA tracings to their corresponding raw signal data segments. Likewise, in some embodiments in which cardiotocograph training data 102 is received as raw signal data, pre-processing component 110 can convert the raw signal data into a corresponding digital graphical FHR-UA tracing prior to input into cardiotocograph pattern identification model 112.

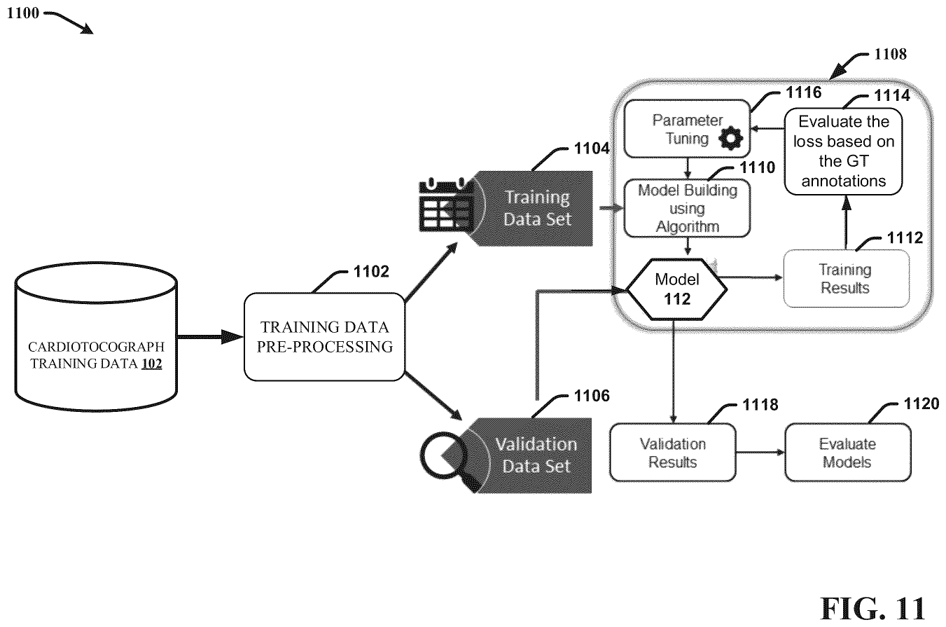

As noted above, in some embodiments, training component 108 can train cardiotocograph pattern identification model 112 to identify distinct patterns in input FHR-UA tracing data samples that represent a window of time, such as 10 minutes or another time interval. In some implementations of these embodiments, pre-processing component 110 can generate these input data samples prior to processing by cardiotocograph pattern identification model 112 by cutting/splicing a continuous FHR-UA tracing for a same subject (e.g., same mother/fetus combination) into sequential segments, wherein each segment represents a defined window of time (e.g., each segment corresponds a 10-minute window of FHR-UA strip data). Pre-processing component 110 can also perform other data pre-processing functions to pre-process the input data prior to input to cardiotocograph pattern identification model 112 (e.g., during training and/or inferencing). For example, pre-processing component 110 can pre-process the input data to fill outliers and missing values (e.g., a process known as data cleansing or data engineering). Training component 108 can further employ a supervised machine learning process to train cardiotocograph pattern identification model 112 to identify the distinct patterns in the input data samples that correspond to defined physiological events (e.g., an FHR acceleration, an FHR deceleration, a period of FHR variability, a contraction, etc.) by employing the manually annotated ground truth data associated with at least some of the input data samples, as described with reference to FIG. 11.

In this regard, FIG. 11 presents an example training process 1100 for training a machine learning model to detect defined patterns in cardiograph data in accordance with one or more embodiments of the disclosed subject matter. In an embodiment, pre-processing component 110 and training component 108 can apply training process 1100 to develop and train one or more cardiotocograph pattern identification models 112. It is to be appreciated that cardiotocograph pattern identification model 112 is illustrated as model 112 in FIG. 11. Repetitive description of like elements employed in respective embodiments is omitted for sake of brevity.

In an embodiment, training process 1100 can initially involve pre-processing cardiotocograph training data 102 by pre-processing component 110 at 1102. The specific pre-processing steps that can be performed at 1102 can vary. For example, in some embodiments, pre-processing component 110 can be configured to segment any continuous strips of FHR-UA data for a same subject into separate input samples that represent short windows of time (e.g., 10 minutes or the like). In other embodiments, cardiotocograph training data 102 can be already formatted as separate input data samples corresponding to defined durations of time (e.g., 10 minutes or the like). The pre-processing at 1102 can also involve converting cardiotocograph training data 102 from one format to another machine-readable format for processing by cardiotocograph pattern identification model 112 (e.g., from signal data to graphical FHR-UA tracing data, or vice versa). The pre-processing at 1102 can also involve data cleansing/engineering to fill outliers and/or missing values.

Once pre-processed, training component 108 can divide cardiotocograph training data 102 into a training data set 1104 and a validation data set 1106. In an embodiment, training data set 1104 can be employed to train and build cardiotocograph pattern identification model 112 in accordance with process 1108. Process 1108 can involve model building at 1110, which can involve applying the input data samples included in training data set 1104 to cardiotocograph pattern identification model 112 to generate training results 1112. The training results 1112 or model output can include information that can identify any distinct patterns in each input data sample (e.g., or no pattern if one is not identified), that correspond to one or more defined physiological events (e.g., an FHR acceleration, an FHR deceleration, a region of marked variability, and/or a contraction), information classifying the specific physiological event corresponding to each pattern, and/or information describing/defining attributes or parameters of the identified pattern. For example, the information describing/defining the attributes or parameters of an identified pattern can include information identifying the specific portions of the data in which the one or more patterns are encompassed (e.g., the FHR data, the UA data, or a combination thereof), the start and stop time points of the one or more patterns, and/or the distribution of measurement values that constitute the pattern (e.g., the specific FHR measurements, the specific UA measurements, and/or the combinations thereof). The format of the model output data can vary. For example, the output data can be formatted in a machine-readable format, in a human readable format, as a graphical FHR-UA tracing with model applied annotation mark ups (e.g., as shown in FIGS. 9 and 10), as text, or another format.

At 1114, process 1108 can involve evaluating the loss based on the ground truth (GT) annotations applied to the corresponding input data samples. The type of loss function employed by training component 108 can vary. For example, the loss function may be based on mean squared error, cross-entropy, hinge loss, and KL divergence, a distance metric or the like. The loss function evaluation at 1114 can generally involve determining a measure of difference in accuracy between training results 1112 and the actual ground truth provided by clinical experts. In accordance with the subject training, training results 1112 can define a pattern identified in a training sample by various metrics that represent the distinct timing and value measurements (e.g., FHR values and/or UA values) that make up the distinct pattern. For example, the pattern can be represented as a graphical distribution of points in space, a reduced dimensionality feature vector, a geometrical shape, a value matrix, or the like. Regardless of the manner in which the pattern is represented/defined, the loss evaluation at 1114 can involve determining differences between the representation of the pattern in the training results and the ground truth representation for the pattern (or no pattern if ground truth indicates no pattern should have been identified).

For example, in some implementations, training component 108 can perform the loss evaluation at 1114, by computing a similarity score between an identified pattern and the corresponding ground truth pattern and determine the degree of loss based on the match similarity score. Training component 108 can compute the similarity score by employing one or more statistical techniques, and/or one or more artificial intelligence techniques. Additionally, or alternatively, training component 108 can compute the similarity score based on a distance metric, such as a Hamming distance, a Jaccard distance/Index, a Dice score/coefficient, or the like.

At 1116, process 1108 can further comprise adjusting the model weights and/or parameters based on the loss to reduce the amount of loss on the next evaluation of the next training data sample. Process 1108 can be performed iteratively until the model loss has stabilized to a defined degree and/or otherwise reached convergence.

Once the cardiotocograph pattern identification model 112 building has progressed to sufficiency on training data set 1104 (e.g., until the model loss has stabilized to a defined degree and/or otherwise reached convergence), the model testing and validation can be performed by employing validation data set 1106. In this regard, training component 108 can apply validation data set 1106 to cardiotocograph pattern identification model 112 to generate validation results 1118, which can be evaluated at 1120 to evaluate the performance accuracy and specificity of cardiotocograph pattern identification model 112. Once the model training (including testing and validation) for cardiotocograph pattern identification model 112 has been completed, cardiotocograph pattern identification model 112 can be deployed in an actual clinical context to automatically identify and classify patterns in new cardiotocograph data that can correspond to defined physiological events and/or conditions.

With reference again to FIG. 1, as noted above, the trained cardiotocograph pattern identification model 112 can be configured to generate a variety of different output data. For example, in an embodiment, the output of cardiotocograph pattern identification model 112 as applied to one input data sample that corresponds to an N minute strip of FHR-UA tracing data (e.g., 10 minutes or another window) can include, but is not limited to: information that identifies any distinct patterns in the data sample that correspond to defined physiological events or conditions (e.g., or no pattern if one is not identified), information that can classify that the physiological event or condition that can correspond to each identified pattern (e.g., an FHR acceleration, an FHR deceleration, a region of marked variability, and/or a contraction), information identifying the data in which the pattern was identified (e.g., the FHR data, the UA data, or a combination thereof), and/or information describing/defining attributes or parameters of the identified patterns (e.g., timing attributes, measurement values, amplitude, frequency attributes, spacing attributes, duration attributes, etc.). In some embodiments, the model output information described above can be presented to clinicians as result data in the form of a report and/or in real-time as the new cardiotocograph data is processed by the cardiotocograph pattern identification model 112 in real-time.

In an embodiment, post-processing component 114 and/or analysis component 118 can, additionally or alternatively, further process and interpret the model output data to generate more accurate and useful results. To facilitate this end, post-processing component 114 and analysis component 118 can employ cardiotocograph interpretation domain knowledge 104 and/or cardiotocograph interpretation schema 116. Cardiotocograph interpretation domain knowledge 104 can include information provided in clinical guidelines, textbooks, articles, and the like that can define rules and guidelines for interpreting cardiotocograph data. This information can include aggregated electronic information from various databases and data sources. Cardiotocograph interpretation schema 116 can include similar rules and guidelines for interpreting cardiograph data, yet tailored specifically for non-limiting system 100 (and other systems described herein), for interpreting the results of cardiotocograph pattern identification model 112. For example, in some embodiments, cardiotocograph interpretation schema 116 can define rules and/or guidelines for removing spurious results by post-processing component 114. With these embodiments, post-processing component 114 can be configured to evaluate the identified patterns and their corresponding physiological events based on defined criteria for valid and invalid patterns provided in cardiotocograph interpretation schema 116, wherein the criteria vary for the different physiological events/condition. For example, the criteria can define thresholds related to timing, duration, frequency, measurement values, and so on. Post-processing component 114 can further remove any identified patterns that fail to pass the defined qualification criteria for the event type from the model results.

In an embodiment, analysis component 118 can also employ cardiotocograph interpretation domain knowledge 104 and/or cardiotocograph interpretation schema 116 to further validate whether an identified pattern indicates a corresponding physiological event or condition occurred or not. For example, in some implementations, based on identification of a pattern by cardiotocograph pattern identification model 112 that correspond to a defined physiological event, the system or analysis component 118 can assume that the event occurred. In other implementations, analysis component 118 can be configured to perform additional evaluation of the pattern information based on cardiotocograph interpretation domain knowledge 104 and/or cardiotocograph interpretation schema 116 to determine with more certainty whether the event or condition did in fact occur. For example, in some implementations, cardiotocograph interpretation schema 116 can define tailored pattern thresholds for different subjects and clinical contexts based on drugs administered, phase of labor, medical condition of the mother, medical condition of the fetus, medical history of the mother, comorbidities, risk level of the mother, and so on. For instance, fetuses with intrauterine growth restriction are unusually susceptible to the effect of hypoxemia, which tends to progress rapidly, which can cause the thresholds for declaring certain patterns as constituting a hypoxemia related event to be lower in this scenario. Thus, analysis component 118 can tailor its evaluation of the identified patterns and their correlation to the occurrence of corresponding physiological events or conditions based on other known information about the subject and the clinical context. Analysis component 118 can also aggregate the model output results for sequential input data samples for sequential time segments to generate a timeline of pattern and event information for the subject that spans across a duration of a time. Analysis component 118 can further evaluate the patterns and event information generated by cardiotocograph pattern identification model 112 longitudinally over time to further clarify the occurrence or non-occurrence of certain events or conditions based on the totality of the aggregated timeline of events and associated patterns.

In an embodiment, analysis component 118 can also employ cardiotocograph interpretation domain knowledge 104 and/or cardiotocograph interpretation schema 116 to determine additional information about the physiological state and condition of the mother and/or fetus based on the output of cardiotocograph pattern identification model 112. For example, in some embodiments, analysis component 118 can employ defined rules and schema regarding how to interpret the model identified patterns corresponding to defined events/conditions to determine additional parameters associated with the defined physiological events and/or conditions. For example, in some embodiments, analysis component 118 can determine the baseline FHR based on the regions of FHR variability determined by cardiotocograph pattern identification model 112 and by employing one or more defined baseline FHR algorithmic formulas defined in cardiotocograph interpretation domain knowledge 104 and/or cardiotocograph interpretation schema 116. Additionally, or alternatively, cardiotocograph pattern identification model 112 can be configured to predict the baseline FHR based on expert applied ground truth data for the baseline FHR, as shown in FIGS. 9 and 10. Analysis component 118 can also determine various other parameters related to the identified patterns and corresponding physiological events/conditions, including but not limited to: timing of FHR acceleration, duration of FHR acceleration, degree of FHR acceleration, type of FHR acceleration, timing of FHR deceleration, duration of FHR deceleration, degree of FHR deceleration, type of FHR deceleration, timing of FHR period of variability, duration of fetal heart period of variability, degree of FHR variability, contraction timing, contraction duration, contraction frequency, and contraction type. In some implementations of these embodiments, cardiotocograph interpretation schema 116 can provide the defined rules, algorithms and/or guidelines employed by analysis component 118 to determine (e.g., calculate) or infer this additional information based on the identified patterns and attributes of the patterns, as well as other input variables related to the patient and the clinical context. Analysis component 118 can also aggregate the model output results for sequential input data samples for sequential time segments to generate a timeline of pattern and event information for the subject that spans across a duration of a time. Analysis component 118 can further summarize the condition and/or status of the mother and/or fetus based on the totality of events and conditions up to the current point of time based on information provided in cardiotocograph interpretation domain knowledge 104 and/or cardiotocograph interpretation schema 116.

In some embodiments, analysis component 118 can employ principles of artificial intelligence to facilitate interpreting the model output data (e.g., the identified patterns and/or their corresponding event classifications) to determine or infer information regarding the clinical condition/state of the mother and/or fetus and/or other relevant parameters associated with the identified patterns and events (e.g., baseline FHR, contraction type, contraction duration, presence of tachysystole, etc.). In certain embodiments, analysis component 118 can include a prediction component that can employ data (e.g., real-time cardiotocograph data, the model output data, cardiotocograph interpretation schema 116 and/or cardiotocograph interpretation domain knowledge 104) to monitor a current state of mother and/or fetus. Analysis component 118 can perform interpretation of cardiotocograph pattern identification model 112 output data explicitly or implicitly. Learning and/or determining inferences by analysis component 118 can facilitate monitoring of one or more patterns in patient flow data. For example, analysis component 118 can employ a probabilistic and/or statistical-based analysis (e.g., factoring into the analysis utilities and costs) to interpret the model output data. Analysis component 118 can employ, for example, an SVM classifier to interpret and classify events and/or conditions associated with the identified patterns. Additionally, or alternatively, analysis component 118 can employ other classification techniques associated with Bayesian networks, decision trees and/or probabilistic classification models. Classifiers employed by analysis component 118 can be explicitly trained (e.g., via a generic training data) as well as implicitly trained (e.g., via observing user behavior, receiving extrinsic information). For example, with respect to SVM's that are well understood, SVM's are configured via a learning or training phase within a classifier constructor and feature selection module. A classifier is a function that maps an input attribute vector, x=(x1, x2, x3, x4, xn), to a confidence that the input belongs to a class—that is, f(x)=confidence(class).

In an aspect, analysis component 118 can include an inference component that can further enhance automated aspects of the model output interpretation utilizing in part inference-based schemes. Analysis component 118 can employ any suitable machine-learning based techniques, statistical-based techniques and/or probabilistic-based techniques. Analysis component 118 can additionally or alternatively employ a reduced set of factors (e.g., an optimized set of factors) to facilitate providing a most accurate machine learning model for predicting events, conditions and parameters associated with the diagnosis and well-being of the fetus and/or the mother based at least in part on the patterns identified by cardiotocograph pattern identification model 112. For example, analysis component 118 can employ expert systems, fuzzy logic, SVMs, HMMs, greedy search algorithms, rule-based systems, Bayesian models (e.g., Bayesian networks), neural networks, other non-linear training techniques, data fusion, utility-based analytical systems, systems employing Bayesian models, etc. In another aspect, analysis component 118 can perform a set of machine learning computations associated with the one or more patterns in the patient cardiotocograph data identified by cardiotocograph pattern identification model 112. For example, analysis component 118 can perform a set of clustering machine learning computations, a set of decision tree machine learning computations, a set of instance-based machine learning computations, a set of regression machine learning computations, a set of regularization machine learning computations, a set of rule learning machine learning computations, a set of Bayesian machine learning computations, a set of deep Boltzmann machine computations, a set of deep belief network computations, a set of convolution neural network computations, a set of stacked auto-encoder computations and/or a set of different machine learning computations. The one or more abnormalities parameters, events and/or conditions determined or inferred by analysis component 118 based at least in part on the output of cardiotocograph pattern identification model 112 can be reported and stored, for example, in cardiotocograph interpretation schema 116 and/or memory 122.

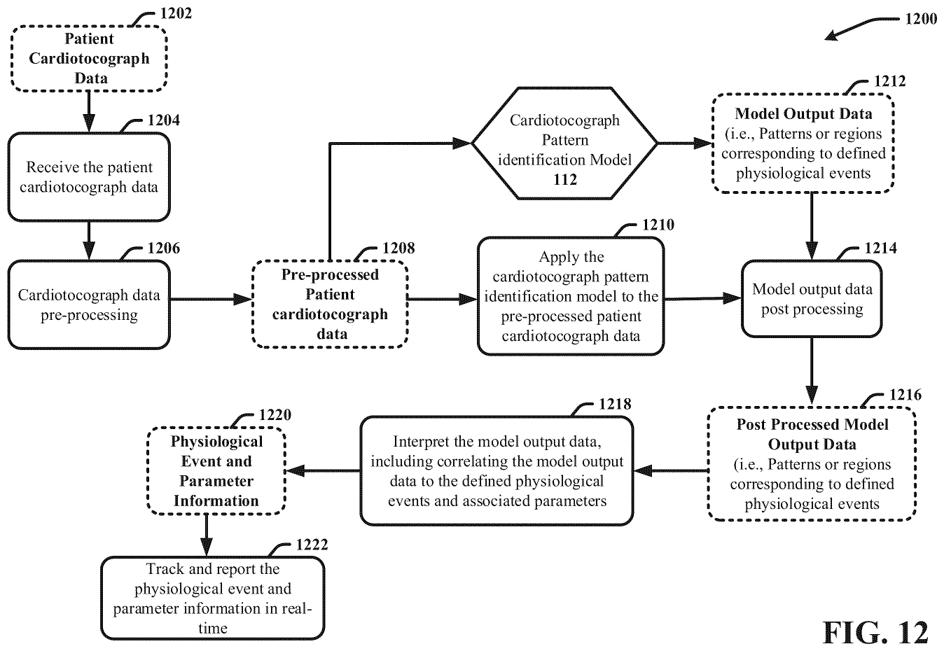

FIG. 12 presents a flow-diagram of an example process 1200 for performing FHR-UA analytics in real-time by employing machine learning techniques in accordance with one or more embodiments of the disclosed subject matter. Process 1200 provides an example process that can be performed by employing cardiotocograph pattern identification model 112 once trained as applied to new cardiotocograph data for a new patient, illustrated in FIG. 12 as patient cardiotocograph data 1202. In some embodiments, patient cardiotocograph data 1202 can correspond to a live stream of cardiotocograph data captured for a mother and fetus over a duration of labor. In other embodiments, cardiotocograph data 1202 can correspond to a set of previously recorded cardiotocograph data, such as that recorded during a monitoring session. Repetitive description of like elements employed in respective embodiments is omitted for sake of brevity.

In accordance with process 1200, at 1204, the system can receive the patient cardiotocograph data 1202. In implementations in which patient cardiotocograph data 1202 corresponds to real-time FHA-UA tracing data, at 1202, this data can be received in real-time. At 1206, the system can pre-process the patient cardiotocograph data 1202 by employing one or more pre-processing techniques disclosed herein to generate pre-processed patient cardiotocograph data 1208. For example, in some implementations, this can involve segmenting the input data into input data samples that can comprise data for defined windows of time (e.g., sequential segments of 10-minute time windows). In some embodiments, the segments can be separated entirely (non-overlapping) back-to-back. For example, a first segment can include times t1-t2, a second segment can include times t2-t3, a third segment can include times t3-t4, and so on. For example, time window t1-t2 can include minutes 0-10, time window t2-t3 can include minutes 11-20, time window t3-14 can include minutes 21-30, and so on. In other embodiments, the segments can be separated in an overlapping fashion. For example, a first segment can include times t1-t2 and correspond to minutes 0-10, a second segment can include times t2-t3 and correspond to minutes 1-11, a third segment can include times t3-t4 and correspond to minutes 2-12 and so on.

At 1210, the system can apply cardiotocograph pattern identification model 112 to the pre-processed patient cardiotocograph data 1208 to generate model output data 1212. For example, model output data 1212 can include identified patterns or regions in the input data samples that correspond to defined physiological events. At 1214, the system can (optionally) perform post-processing on the model output data (e.g., by employing post-processing component 114), such as to remove spurious results, convert the output data into another format, or the like. The resulting post-processed model output data is represented in process 1200 as post-processed model output data 1216. At 1218, the system can interpret the model output data (e.g., post-processed model output data 1216) by employing analysis component 118, cardiotocograph interpretation schema 116 and/or cardiotocograph interpretation domain knowledge 104. This can involve for example, correlating the model output data to the defined physiological events and associated parameters (e.g., determining the baseline FHR, determining the contraction duration, determining contraction type, determining tachysystole presence, and so on). The final output of cardiotocograph pattern identification model 112 and analysis component 118 processing can include physiological event and parameter information 1220 regarding the identified physiological events that have occurred and relevant parameters and associated diagnosis/condition of the mother and/or fetus. At 1222, the system can further track and report the physiological event and parameter information, which can be performed in real-time (e.g., over the course of labor) in implementations in which the patient cardiotocograph data 1202 is received in real-time.

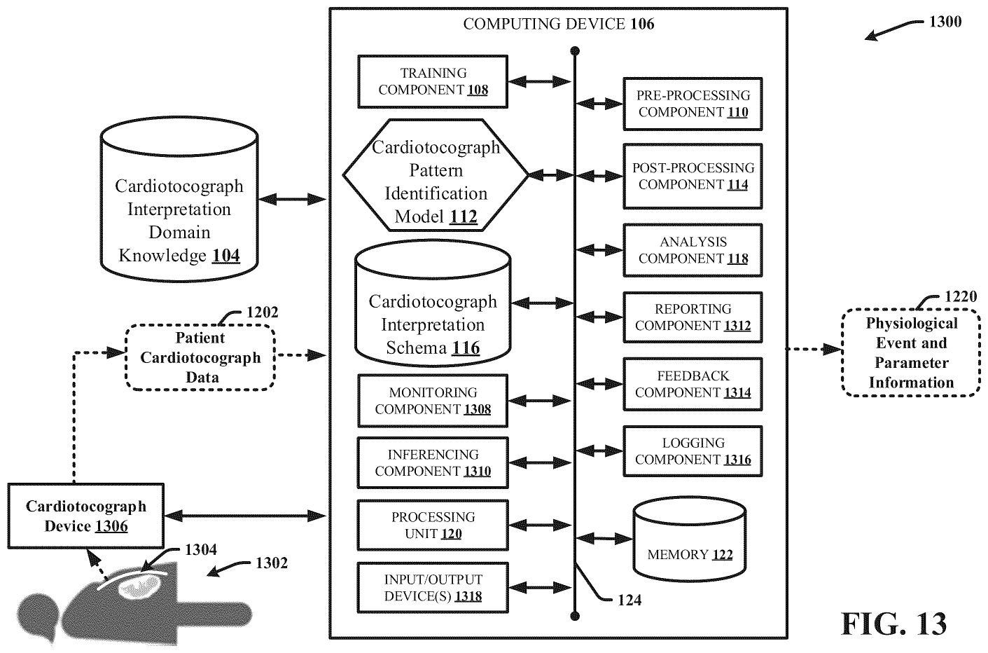

FIG. 13 illustrates an example system 1300 for performing FHR-UA analytics in real-time by employing machine learning techniques in accordance with one or more embodiments of the disclosed subject matter. System 1300 provides an example system for performing process 1200 and similar processes by employing one or more trained cardiotocograph pattern identification models 112. System 1300 can comprise the same or similar components as non-limiting system 100 with the addition of monitoring component 1308, inferencing component 1310, reporting component 1312, feedback component 1314, logging component 1316 and input/output devices 1318 to the computing device 106. System 1300 can further comprise cardiotocograph device 1306 that can be coupled to patient 1302 and the computing device 106. Repetitive description of like elements employed in respective embodiments is omitted for sake of brevity.

In accordance with system 1300, patient 1302 can include a mother and fetus. Cardiotocograph device 1306 can be electrically and operatively coupled to the patient via one or more sensors 1304 (e.g., transducers or the like) that supply FHR and UA reading signals to cardiotocograph device 1306 which convert the signals into time/measurement values and generate the corresponding FHR-UA tracing data for the patient in real-time. Cardiotocograph device 1306 further provides the raw signal data and/or the FHR-UA tracing data to computing device 106 for processing thereof. In the embodiment shown, the raw signal data and/or the FHR-UA tracing data is represented as patient cardiotocograph data 1202.

In an embodiment, monitoring component 1308 can receive patient cardiotocograph data 1202 from cardiotocograph device 1306. For example, in implementations in which patient 1302 can be hooked up to cardiotocograph device 1306 over a duration of labor, and monitoring component 1308 can receive patient cardiotocograph data 1202 in real-time over the duration of labor. Inferencing component 1310 can further apply cardiotocograph pattern identification model 112 to patient cardiotocograph data 1202 as it is received to generate the model output data (e.g., model output data 1212). In this regard, the model output data can include information that can identify patterns in the input data that correspond to defined physiological events associated with the fetus and/or the mother. Analysis component 118 can further determine occurrence of the defined physiological events based on the patterns in the new cardiotocograph data. Analysis component 118 can also determine various other parameters related to the identified patterns and corresponding physiological events/conditions by employing cardiotocograph interpretation schema 116 and/or cardiotocograph interpretation domain knowledge 104 via the techniques described above. For example, this additional information can include, but is not limited to: baseline FHR, timing of FHR acceleration, duration of FHR acceleration, degree of FHR acceleration, type of FHR acceleration, timing of FHR deceleration, duration of FHR deceleration, degree of FHR deceleration, type of FHR deceleration, timing of FHR period of variability, duration of fetal heart period of variability, degree of FHR variability, contraction timing, contraction duration, contraction frequency, and contraction type.

In an embodiment, reporting component 1312 can further report the results of cardiotocograph pattern identification model 112 and/or analysis component 118 in real-time. In this regard, reporting component 1312 can report physiological event and parameter information 1220 regarding the identified physiological events that occurred, the related parameters and related diagnosis and condition information to one or more clinicians involved in the treatment of the patient 1302 via one or more suitable input/output device 1318 (e.g., via display, a speaker, or the like). Some suitable input/output devices can be described in FIG. 17 with reference to output device 1736 and input device 1728.

In an embodiment, feedback component 1314 can further facilitate receiving feedback information from the one or more clinicians regarding accuracy of the event information and the parameter information. For example, in some implementations, reporting component 1312 can provide the physiological event and parameter information 1220 to the clinicians via an interactive graphical user interface (GUI) that includes a feedback mechanism for receiving user input accepting or rejecting the results. The feedback mechanism can also allow the user to provide input identify errors in the results and/or providing corrections to the results. In an embodiment, logging component 1316 can further log the feedback information with the new cardiograph data recorded for patient 1302 in an indexed data structure, wherein training component 108 can further employ the feedback information in association with retraining and refining cardiotocograph pattern identification model 112 over time (e.g., offline).

As described herein, a real-time computer system can be defined as a computer system that can perform its functions and respond to external, asynchronous events within a defined, predictable (or deterministic) amount of time. A real-time computer system such as system 1300 can typically control a process (e.g., monitoring patient cardiotocograph data 1202 and, identifying patterns in the data corresponding to physiological events and conditions, and reporting the data) by recognizing and responding to discrete events within predictable time intervals, and by processing and storing large amounts of data acquired from the controlled system (e.g., the cardiotocograph device 1306). Response time and data throughput requirements can depend on the specific real-time application, data acquisition and critical nature of the type of decision support provided. In this regard, the term “real-time” as used herein with reference to processing and generating information by computing device 106 refers to performance of these actions within a defined or predictable amount of time (e.g., a few seconds, less than 10 seconds, less than a minute, etc.) between reception of patient cardiotocograph data 1202 by monitoring component 1308.

The deployment architecture of system 1300 (and other systems described herein) can vary. In some embodiments, the components of computing device 106 can be deployed at and executed by a single computing device (e.g., real or virtual) operatively coupled to processor 120 and memory 122. In some implementations of these embodiments, computing device 106 and cardiotocograph device 706 can be physically, operatively, and/or communicatively coupled. In other embodiments, one or more components of computing device 106 can be deployed at two or more separate communicatively coupled computing devices operating in a distributed computing environment. The separate computing devices can be communicatively coupled via one or more wired or wireless communication networks. Various alternative deployment architecture variations can also be used.

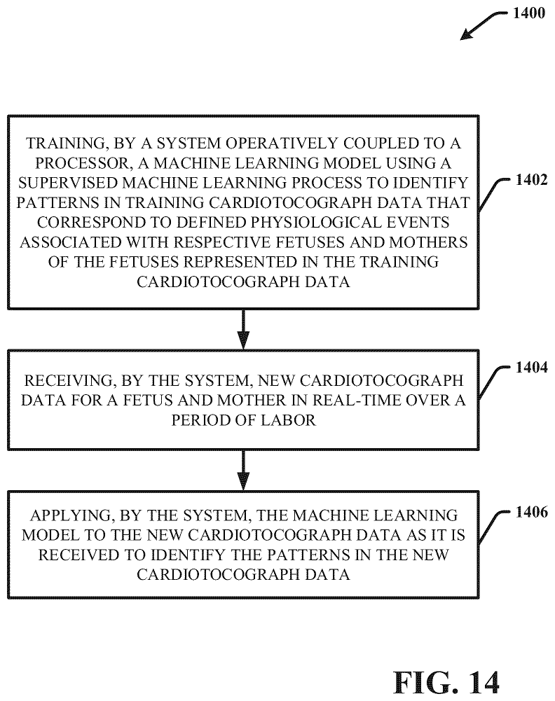

FIG. 14 presents a high-level flow diagram of an example computer-implemented process 1400 to perform FHR analytics by employing machine learning techniques in accordance with one or more embodiments of the disclosed subject matter. Repetitive description of like elements employed in respective embodiments is omitted for sake of brevity.

In accordance with computer-implemented process 1400, at 1402 a system operatively coupled to a processor (e.g., non-limiting system 100, system 1300 and the like) can train (e.g., by employing training component 108) a machine learning model (e.g., cardiotocograph pattern identification model 112) by employing a supervised machine learning process (e.g., training process 1100 or the like) to identify patterns in training cardiotocograph data (e.g., cardiotocograph training data 102) that correspond to defined physiological events associated with respective fetuses and mothers of the fetuses represented in the training cardiotocograph data. For example, the patterns can correspond to an FHR acceleration, an FHR deceleration, a uterine contraction, a period of FHR variability, and a uterine tachysystole. Other physiological events that can be reflected by defined patterns in the cardiotocograph data are also envisioned. Once the model training has been completed, at 1404, the system can receive new cardiotocograph data for a fetus and mother (e.g., patient cardiotocograph data 1202) in real-time over a period of labor (e.g., via monitoring component 1308). At 1406, the system can apply (e.g., via inferencing component 1310) the machine learning model to the new cardiotocograph data, as it is received, to identify the patterns in the new cardiotocograph data.

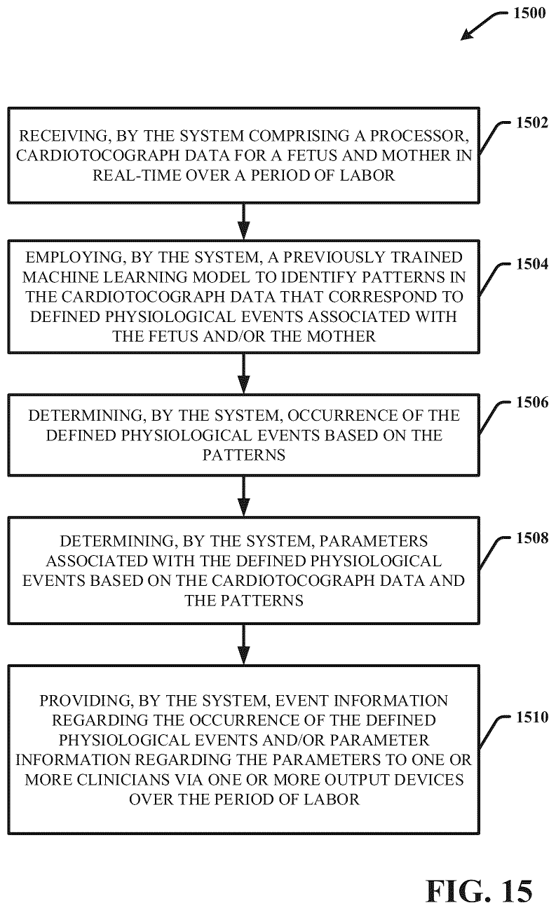

FIG. 15 presents a high-level flow diagram of another example computer-implemented process 1500 to perform FHR analytics by employing machine learning techniques in accordance with one or more embodiments of the disclosed subject matter. Repetitive description of like elements employed in respective embodiments is omitted for sake of brevity.

In accordance with computer-implemented process 1500, at 1502 a system operatively coupled to a processor (e.g., system 1300 and the like) can receive cardiotocograph data for a fetus and mother (e.g., patient cardiotocograph data 1202) in real-time over a period of labor (e.g., via monitoring component 1308). At 1504, the system can employ a previously trained machine learning model (e.g., cardiotocograph pattern identification model 112) to identify patterns in the cardiotocograph data that can correspond to defined physiological events associated with the fetus and/or the mother (e.g., via inferencing component 1310). At 1506, the system can determine occurrence of the defined physiological events based on the patterns (e.g., via inferencing component 1310 and/or analysis component 118). For example, in some embodiments, cardiotocograph pattern identification model 112 can be configured to directly correlate a pattern in the cardiotocograph data to one or more defined physiological events, such as an FHR acceleration, an FHR deceleration, a period of FHR variability or a contraction. In some implementations of these embodiments, in response to detection of a pattern that corresponds to such an event, inferencing component 1310 can generate an output that can indicate the event that occurred and the timing of occurrence. Additionally, or alternatively, analysis component 118 can further analyze the detected patterns in view of cardiotocograph interpretation schema 116 and employ one or more additional machine learning and/or rule-based algorithms to facilitate correlating the detected patterns to occurrence of the defined physiological events.

At 1508, the system can determine parameters associated with the defined physiological events based on the cardiotocograph data and the patterns (e.g., via analysis component 118 and by employing cardiotocograph interpretation schema 116). For example, the parameters may include, but are not limited to: baseline FHR, timing of FHR acceleration, duration of FHR acceleration, degree of FHR acceleration, type of FHR acceleration, timing of FHR deceleration, duration of FHR deceleration, degree of FHR deceleration, type of FHR deceleration, timing of FHR period of variability, duration of fetal heart period of variability, degree of FHR variability, contraction timing, contraction duration, contraction frequency, and contraction type. At 1510, the system can provide (e.g., via reporting component 1312) event information regarding the occurrence of the defined physiological events and/or parameter information regarding the parameters (e.g., physiological event and parameter information 1220) to one or more clinicians via one or more output devices (e.g., via a display monitor, a speaker, or the like) over the period of labor.

FIGS. 14 and 15 relate to embodiments of the present disclosure that can provide a deep learning-based approach for fetal heart rate analytics. The disclosed AI-based approach can involve a deep learning model trained on annotated data to analyze FHR-UA strips. This data driven approach can ensure that the contextual clinical understanding can be accurately captured during inferencing due to the model being trained by employing FHR-UA strip data annotated by experts as the ground truth.

In this regard, in an embodiment, a system is provided that can comprise a processor that can execute computer-executable components stored in memory. The computer-executable components can comprise a training component that can train a machine learning model by employing a supervised machine learning process to identify patterns in training cardiotocograph data that can correspond to defined physiological events associated with respective fetuses and mothers of the fetuses represented in the training cardiotocograph data. The computer-executable components can further comprise a monitoring component that can receive new cardiotocograph data for a fetus and mother in real-time over a period of labor, and an inferencing component that can apply the machine learning model to the new cardiotocograph data, as it is received, to identify the patterns in the new cardiotocograph data.

In an embodiment, at least some of the training cardiotocograph data can comprise annotated cardiotocography data annotated with information identifying the patterns and the defined physiological events that can respectively correspond to the patterns, wherein the supervised machine learning process can comprise employing the annotated cardiotocograph data as ground truth. In some implementations, the annotated cardiotocograph data can comprise annotations applied to graphical cardiotocograph strips generated from the training cardiotocograph data. In an embodiment, the computer-executable components can further comprise a pre-processing component that can convert the annotated cardiotocograph data into a machine-readable format for processing by the machine learning model.

In an embodiment, the computer-executable components can further comprise an analysis component that can determine occurrence of the defined physiological events based on the patterns in the new cardiotocograph data, and a reporting component that can provide event information regarding the occurrence of the defined physiological events to one or more clinicians via one or more output devices over the period of labor. For example, the defined physiological events can include, but are not limited to, an FHR acceleration, an FHR deceleration, a uterine contraction, a period of FHR variability, and a uterine tachysystole. In some implementations, the analysis component can further determine parameters associated with the defined physiological events based on the new cardiotocograph data and the patterns in the new cardiotocograph data, and the reporting component can further provide parameter information regarding the parameters associated with the defined physiological events with the event information. For example, the parameters can include, but are not limited to: baseline FHR, timing of FHR acceleration, duration of FHR acceleration, degree of FHR acceleration, type of FHR acceleration, timing of FHR rate deceleration, duration of FHR deceleration, degree of FHR deceleration, type of FHR rate deceleration, timing of FHR rate period of variability, duration of FHR period of variability, degree of FHR variability, contraction timing, contraction duration, contraction frequency, and contraction type.

In some implementations, the computer-executable components can further comprise a feedback component that can facilitate receiving feedback information from the one or more clinicians regarding accuracy of the event information and the parameter information, and a logging component that can log the feedback information with the new cardiograph data in an indexed data structure, wherein the training component can further employ the feedback information in association with retraining and refining the machine learning model.

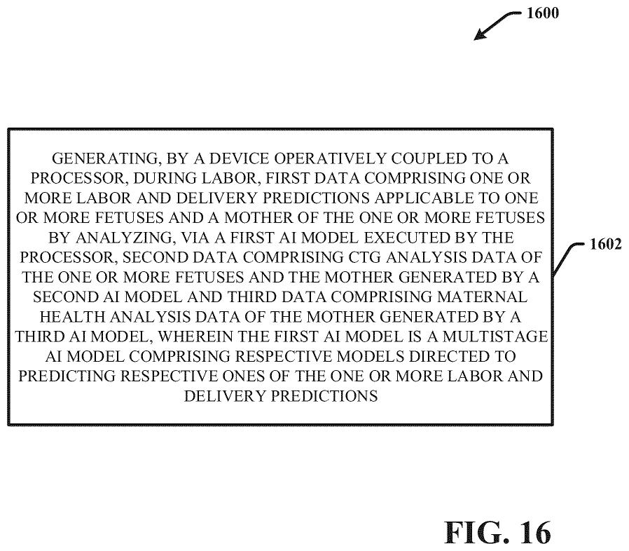

FIG. 16 illustrates a flow diagram of an example, non-limiting method 1600 that can generate labor and delivery predictions in accordance with one or more embodiments of the disclosed subject matter. Repetitive description of like elements employed in respective embodiments is omitted for sake of brevity.

At 1602, non-limiting method 1600 can comprise generating (e.g., by AI model 126), by a device operatively coupled to a processor, during labor, first data comprising one or more labor and delivery predictions applicable to one or more fetuses and a mother of the one or more fetuses by analyzing, via a first AI model executed by the processor, second data comprising CTG analysis data of the one or more fetuses and the mother generated by a second AI model and third data comprising maternal health analysis data of the mother generated by a third AI model, wherein the first AI model is a multistage AI model comprising respective models directed to predicting respective ones of the one or more labor and delivery predictions.

### Example Operating Environment

One or more embodiments can be a system, a method, and/or a computer program product at any possible technical detail level of integration. The computer program product can include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium can be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network can comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention can be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, procedural programming languages, such as the “C” programming language or similar programming languages, and machine-learning programming languages such as like CUDA, Python, Tensorflow, PyTorch, and the like. The computer readable program instructions can execute entirely on the user's computer, partly on the user's computer, as a stand-alone software package, partly on the user's computer and partly on a remote computer or entirely on the remote computer or server using suitable processing hardware. In the latter scenario, the remote computer can be connected to the user's computer through any type of network, including a local area network (LAN) or a wide area network (WAN), or the connection can be made to an external computer (for example, through the Internet using an Internet Service Provider). In various embodiments involving machine-learning programming instructions, the processing hardware can include one or more graphics processing units (GPUs), central processing units (CPUs), and the like. For example, one or more of the disclosed machine-learning models (e.g., the cardiotocograph pattern identification model 112) may be written in a suitable machine-learning programming language and executed via one or more GPUs, CPUs or combinations thereof. In some embodiments, electronic circuitry including, for example, programmable logic circuitry, field-programmable gate arrays (FPGA), or programmable logic arrays (PLA) can execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It can be understood that each block of the flowchart illustrations and/or block diagrams, and combinations of blocks in the flowchart illustrations and/or block diagrams, can be implemented by computer readable program instructions.

These computer readable program instructions can be provided to a processor of a general-purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions can also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions can also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams can represent a module, segment, or portion of instructions, which comprises one or more executable instructions for implementing the specified logical function(s). In some alternative implementations, the functions noted in the blocks can occur out of the order noted in the Figures. For example, two blocks shown in succession may, in fact, be executed substantially concurrently, or the blocks can sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

In connection with FIG. 17, the systems and processes described below can be embodied within hardware, such as a single integrated circuit (IC) chip, multiple ICs, an application specific integrated circuit (ASIC), or the like. Further, the order in which some or all of the process blocks appear in each process should not be deemed limiting. Rather, it should be understood that some of the process blocks can be executed in a variety of orders, not all of which can be explicitly illustrated herein.

With reference to FIG. 17, an example environment 1700 for implementing various aspects of the claimed subject matter includes a computer 1702. The computer 1702 includes a processing unit 1704, a system memory 1706, a codec 1735, and a system bus 1708. The system bus 1708 couples system components including, but not limited to, the system memory 1706 to the processing unit 1704. The processing unit 1704 can be any of various available processors. Dual microprocessors, one or more GPUs, CPUs, and other multiprocessor architectures also can be employed as the processing unit 1704.

The system bus 1708 can be any of several types of bus structure(s) including the memory bus or memory controller, a peripheral bus or external bus, or a local bus using any variety of available bus architectures including, but not limited to, Industrial Standard Architecture (ISA), Micro-Channel Architecture (MSA), Extended ISA (EISA), Intelligent Drive Electronics (IDE), VESA Local Bus (VLB), Peripheral Component Interconnect (PCI), Card Bus, Universal Serial Bus (USB), Advanced Graphics Port (AGP), Personal Computer Memory Card International Association bus (PCMCIA), Firewire (IEEE 1394), and Small Computer Systems Interface (SCSI).

The system memory 1706 includes volatile memory 1726 and non-volatile memory 1712, which can employ one or more of the disclosed memory architectures, in various embodiments. The basic input/output system (BIOS), containing the basic routines to transfer information between elements within the computer 1702, such as during start-up, is stored in non-volatile memory 1712. In addition, according to present innovations, codec 1735 can include at least one of an encoder or decoder, wherein the at least one of an encoder or decoder can consist of hardware, software, or a combination of hardware and software. Although, codec 1735 is depicted as a separate component, codec 1735 can be contained within non-volatile memory 1712. By way of illustration, and not limitation, non-volatile memory 1712 can include read only memory (ROM), programmable ROM (PROM), electrically programmable ROM (EPROM), electrically erasable programmable ROM (EEPROM), Flash memory, 3D Flash memory, or resistive memory such as resistive random access memory (RRAM). Non-volatile memory 1712 can employ one or more of the disclosed memory devices, in at least some embodiments. Moreover, non-volatile memory 1712 can be computer memory (e.g., physically integrated with computer 1702 or a mainboard thereof), or removable memory. Examples of suitable removable memory with which disclosed embodiments can be implemented can include a secure digital (SD) card, a compact Flash (CF) card, a universal serial bus (USB) memory stick, or the like. Volatile memory 1726 includes random access memory (RAM), which acts as external cache memory, and can also employ one or more disclosed memory devices in various embodiments. By way of illustration and not limitation, RAM is available in many forms such as static RAM (SRAM), dynamic RAM (DRAM), synchronous DRAM (SDRAM), double data rate SDRAM (DDR SDRAM), and enhanced SDRAM (ESDRAM) and so forth.

Computer 1702 can also include removable/non-removable, volatile/non-volatile computer storage medium. FIG. 17 illustrates, for example, disk storage 1714. Disk storage 1714 includes, but is not limited to, devices like a magnetic disk drive, solid state disk (SSD), flash memory card, or memory stick. In addition, disk storage 1714 can include storage medium separately or in combination with other storage medium including, but not limited to, an optical disk drive such as a compact disk ROM device (CD-ROM), CD recordable drive (CD-R Drive), CD rewritable drive (CD-RW Drive) or a digital versatile disk ROM drive (DVD-ROM). To facilitate connection of the disk storage 1714 to the system bus 1708, a removable or non-removable interface is typically used, such as interface 1716. It is appreciated that disk storage 1714 can store information related to a user. Such information might be stored at or provided to a server or to an application running on a user device. In one embodiment, the user can be notified (e.g., by way of output device(s) 1736) of the types of information that are stored to disk storage 1714 or transmitted to the server or application. The user can be provided the opportunity to opt-in or opt-out of having such information collected or shared with the server or application (e.g., by way of input from input device(s) 1728).

It is to be appreciated that FIG. 17 describes software that acts as an intermediary between users and the basic computer resources described in the suitable operating environment 1700. Such software includes an operating system 1718. Operating system 1718, which can be stored on disk storage 1714, acts to control and allocate resources of the computer 1702. Applications 1720 take advantage of the management of resources by operating system 1718 through program modules 1724, and program data 1710, such as the boot/shutdown transaction table and the like, stored either in system memory 1706 or on disk storage 1714. It is to be appreciated that the claimed subject matter can be implemented with various operating systems or combinations of operating systems.

A user enters commands or information into the computer 1702 through input device(s) 1728. Input devices 1728 include, but are not limited to, a pointing device such as a mouse, trackball, stylus, touch pad, keyboard, microphone, joystick, game pad, satellite dish, scanner, TV tuner card, digital camera, digital video camera, web camera, and the like. These and other input devices connect to the processing unit 1704 through the system bus 1708 via interface port(s) 1730. Interface port(s) 1730 include, for example, a serial port, a parallel port, a game port, and a universal serial bus (USB). Output device(s) 1736 use some of the same type of ports as input device(s) 1728. Thus, for example, a USB port can be used to provide input to computer 1702 and to output information from computer 1702 to an output device 1736. Output adapter 1734 is provided to illustrate that there are some output devices 1736 like monitors, speakers, and printers, among other output devices 1736, which require special adapters. The output adapters 1734 include, by way of illustration and not limitation, video and sound cards that provide a means of connection between the output device 1736 and the system bus 1708. It should be noted that other devices or systems of devices provide both input and output capabilities such as remote computer(s) 1738.

Computer 1702 can operate in a networked environment using logical connections to one or more remote computers, such as remote computer(s) 1738. The remote computer(s) 1738 can be a personal computer, a server, a router, a network PC, a workstation, a microprocessor based appliance, a peer device, a smart phone, a tablet, or other network node, and typically includes many of the elements described relative to computer 1702. For purposes of brevity, only a memory storage device 1740 is illustrated with remote computer(s) 1738. Remote computer(s) 1738 is logically connected to computer 1702 through a network interface 1742 and then connected via communication connection(s) 1744. Network interface 1742 encompasses wire or wireless communication networks such as local-area networks (LAN) and wide-area networks (WAN) and cellular networks. LAN technologies include Fiber Distributed Data Interface (FDDI), Copper Distributed Data Interface (CDDI), Ethernet, Token Ring and the like. WAN technologies include, but are not limited to, point-to-point links, circuit switching networks like Integrated Services Digital Networks (ISDN) and variations thereon, packet switching networks, and Digital Subscriber Lines (DSL).

Communication connection(s) 1744 refers to the hardware/software employed to connect the network interface 1742 to the bus 1708. While communication connection 1744 is shown for illustrative clarity inside computer 1702, it can also be external to computer 1702. The hardware/software necessary for connection to the network interface 1742 includes, for exemplary purposes only, internal and external technologies such as, modems including regular telephone grade modems, cable modems and DSL modems, ISDN adapters, and wired and wireless Ethernet cards, hubs, and routers.

While the subject matter has been described above in the general context of computer-executable instructions of a computer program product that runs on a computer and/or computers, those skilled in the art will recognize that this disclosure also can or can be implemented in combination with other program modules. Generally, program modules include routines, programs, components, data structures, etc. that perform particular tasks and/or implement particular abstract data types. Moreover, those skilled in the art will appreciate that the inventive computer-implemented methods can be practiced with other computer system configurations, including single-processor or multiprocessor computer systems, mini-computing devices, mainframe computers, as well as computers, hand-held computing devices (e.g., PDA, phone), microprocessor-based or programmable consumer or industrial electronics, and the like. The illustrated aspects can also be practiced in distributed computing environments where tasks are performed by remote processing devices that are linked through a communications network. However, some, if not all aspects of this disclosure can be practiced on stand-alone computers. In a distributed computing environment, program modules can be located in both local and remote memory storage devices.

As used in this application, the terms “component,” “system,” “platform,” “interface,” and the like, can refer to and/or can include a computer-related entity or an entity related to an operational machine with one or more specific functionalities. The entities disclosed herein can be either hardware, a combination of hardware and software, software, or software in execution. For example, a component can be, but is not limited to being, a process running on a processor, a processor, an object, an executable, a thread of execution, a program, and/or a computer. By way of illustration, both an application running on a server and the server can be a component. One or more components can reside within a process and/or thread of execution and a component can be localized on one computer and/or distributed between two or more computers. In another example, respective components can execute from various computer readable media having various data structures stored thereon. The components can communicate via local and/or remote processes such as in accordance with a signal having one or more data packets (e.g., data from one component interacting with another component in a local system, distributed system, and/or across a network such as the Internet with other systems via the signal). As another example, a component can be an apparatus with specific functionality provided by mechanical parts operated by electric or electronic circuitry, which is operated by a software or firmware application executed by a processor. In such a case, the processor can be internal or external to the apparatus and can execute at least a part of the software or firmware application. As yet another example, a component can be an apparatus that provides specific functionality through electronic components without mechanical parts, wherein the electronic components can include a processor or other means to execute software or firmware that confers at least in part the functionality of the electronic components. In an aspect, a component can emulate an electronic component via a virtual machine, e.g., within a cloud computing system.

In addition, the term “or” is intended to mean an inclusive “or” rather than an exclusive “or.” That is, unless specified otherwise, or clear from context, “X employs A or B” is intended to mean any of the natural inclusive permutations. That is, if X employs A; X employs B; or X employs both A and B, then “X employs A or B” is satisfied under any of the foregoing instances. Moreover, articles “a” and “an” as used in the subject specification and annexed drawings should generally be construed to mean “one or more” unless specified otherwise or clear from context to be directed to a singular form. As used herein, the terms “example” and/or “exemplary” are utilized to mean serving as an example, instance, or illustration and are intended to be non-limiting. For the avoidance of doubt, the subject matter disclosed herein is not limited by such examples. In addition, any aspect or design described herein as an “example” and/or “exemplary” is not necessarily to be construed as preferred or advantageous over other aspects or designs, nor is it meant to preclude equivalent exemplary structures and techniques known to those of ordinary skill in the art.

As it is employed in the subject specification, the term “processor” can refer to substantially any computing processing unit or device comprising, but not limited to, single-core processors; single-processors with software multithread execution capability; multi-core processors; multi-core processors with software multithread execution capability; multi-core processors with hardware multithread technology; parallel platforms; and parallel platforms with distributed shared memory. Additionally, a processor can refer to an integrated circuit, an application specific integrated circuit (ASIC), a digital signal processor (DSP), a field programmable gate array (FPGA), a programmable logic controller (PLC), a complex programmable logic device (CPLD), a discrete gate or transistor logic, discrete hardware components, or any combination thereof designed to perform the functions described herein. Further, processors can exploit nano-scale architectures such as, but not limited to, molecular and quantum-dot based transistors, switches and gates, in order to optimize space usage or enhance performance of user equipment. A processor can also be implemented as a combination of computing processing units. In this disclosure, terms such as “store,” “storage,” “data store,” data storage,” “database,” and substantially any other information storage component relevant to operation and functionality of a component are utilized to refer to “memory components,” entities embodied in a “memory,” or components comprising a memory. It is to be appreciated that memory and/or memory components described herein can be either volatile memory or nonvolatile memory, or can include both volatile and nonvolatile memory. By way of illustration, and not limitation, nonvolatile memory can include read only memory (ROM), programmable ROM (PROM), electrically programmable ROM (EPROM), electrically erasable ROM (EEPROM), flash memory, or nonvolatile random access memory (RAM) (e.g., ferroelectric RAM (FeRAM). Volatile memory can include RAM, which can act as external cache memory, for example. By way of illustration and not limitation, RAM is available in many forms such as synchronous RAM (SRAM), dynamic RAM (DRAM), synchronous DRAM (SDRAM), double data rate SDRAM (DDR SDRAM), enhanced SDRAM (ESDRAM), Synchlink DRAM (SLDRAM), direct Rambus RAM (DRRAM), direct Rambus dynamic RAM (DRDRAM), and Rambus dynamic RAM (RDRAM). Additionally, the disclosed memory components of systems or computer-implemented methods herein are intended to include, without being limited to including, these and any other suitable types of memory.

What has been described above include mere examples of systems and computer-implemented methods. It is, of course, not possible to describe every conceivable combination of components or computer-implemented methods for purposes of describing this disclosure, but one of ordinary skill in the art can recognize that many further combinations and permutations of this disclosure are possible. Furthermore, to the extent that the terms “includes,” “has,” “possesses,” and the like are used in the detailed description, claims, appendices and drawings such terms are intended to be inclusive in a manner similar to the term “comprising” as “comprising” is interpreted when employed as a transitional word in a claim. The descriptions of the various embodiments have been presented for purposes of illustration, but are not intended to be exhaustive or limited to the embodiments disclosed. Many modifications and variations can be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

What is claimed is:

1. A system, comprising:
a processor that executes computer-executable components stored in memory, wherein the computer-executable components comprise:
a first artificial intelligence (AI) model that generates first data comprising one or more labor and delivery predictions applicable to one or more fetuses and a mother of the one or more fetuses, during labor, by analyzing second data comprising cardiotocography (CTG) analysis data of the one or more fetuses and the mother generated by a second AI model and third data comprising maternal health analysis data of the mother generated by a third AI model, wherein the first AI model is a multistage AI model comprising respective models directed to predicting respective ones of the one or more labor and delivery predictions.

2. The system of claim 1, wherein the first data comprises one or more types of data selected from a group comprising fetal hypoxia, fetal acidemia, fetal acidosis, labor progression indicating a C-section, cervical dilation progression, a postpartum hemorrhage risk assessment, a preeclampsia prediction, a labor induction recommendation, a sepsis possibility, and one or more additional labor and delivery predictions.

3. The system of claim 1, wherein the second AI model generates the second data by processing fetal heart rate (FHR) data of the one or more fetuses and uterine activity (UA) data of the mother, and wherein the second data comprises one or more types of data selected from a group consisting of an FHR baseline value calculation, an FHR acceleration, an FHR deceleration, a contraction, an FHR variability value calculation, fetal tracing classification, and one or more additional CTG analysis data types.

4. The system of claim 1, wherein the third AI model generates the third data by employing rule-based algorithms to process electronic medical records (EMRs) and health parameters of the mother, and wherein the third data comprises one or more types of data selected from a group consisting of maternal health related risk factors, pregnancy related complications, dystocia, genetic disorders and one or more additional maternal health analysis data types.

5. The system of claim 1, wherein the second data and the third data are generated during fetal monitoring of the one or more fetuses and maternal monitoring of the mother, and wherein the second data and the third data are available for analysis by the first AI model during the fetal monitoring of the one or more fetuses and the maternal monitoring of the mother.

6. The system of claim 1, further comprising:
a training component that trains the first AI model to generate the first data by employing the second data and the third data as input data to the first AI model and employing known labor and delivery predictions corresponding to the input data as output data for the first AI model, and that trains the second AI model by employing a supervised machine learning process to identify patterns in training cardiotocograph data that correspond to defined physiological events associated with respective fetuses and mothers of the fetuses represented in the training cardiotocograph data.

7. The system of claim 6, wherein at least some of the training cardiotocograph data comprises annotated cardiotocograph data annotated with information identifying the patterns and the defined physiological events that respectively correspond to the patterns, and wherein the supervised machine learning process comprises employing the annotated cardiotocograph data as ground truth.

8. The system of claim 1, further comprising:
an alert component that generates an alert in response to the first data being indicative of an emergency situation.

9. The system of claim 1, further comprising:
an output component that displays the first data at a device accessible to an entity for further analysis of the first data to identify actions to be executed by the entity for safe delivery of the one or more fetuses.

10. A computer-implemented method, comprising:
generating, by a device operatively coupled to a processor, during labor, first data comprising one or more labor and delivery predictions applicable to one or more fetuses and a mother of the one or more fetuses by analyzing, via a first AI model executed by the processor, second data comprising CTG analysis data of the one or more fetuses and the mother generated by a second AI model and third data comprising maternal health analysis data of the mother generated by a third AI model, wherein the first AI model is a multistage AI model comprising respective models directed to predicting respective ones of the one or more labor and delivery predictions.

11. The computer-implemented method of claim 10, wherein the first data comprises one or more types of data selected from a group comprising fetal hypoxia, fetal acidemia, fetal acidosis, labor progression indicating a C-section, cervical dilation progression, a postpartum hemorrhage risk assessment, a preeclampsia prediction, a labor induction recommendation, a sepsis possibility, and one or more additional labor and delivery predictions.

12. The computer-implemented method of claim 10, further comprising:
generating, by the device, via the second AI model executed by the processor, the second data by processing FHR data of the one or more fetuses and UA data of the mother, and wherein the second data comprises one or more types of data selected from a group consisting of an FHR baseline value calculation, an FHR acceleration, an FHR deceleration, a contraction, an FHR variability value calculation, fetal tracing classification, and one or more additional CTG analysis data types.

13. The computer-implemented method of claim 10, further comprising:
generating, by the device, via the third AI model executed by the processor, the third data by employing rule-based algorithms to process EMRs and health parameters of the mother, wherein the third data comprises one or more types of data selected from a group consisting of maternal health related risk factors, pregnancy related complications, dystocia, genetic disorders and one or more additional maternal health analysis data types.

14. The computer-implemented method of claim 10, wherein the second data and the third data are generated during fetal monitoring of the one or more fetuses and maternal monitoring of the mother, and wherein the second data and the third data are available for analysis by the first AI model during the fetal monitoring of the one or more fetuses and the maternal monitoring of the mother.

15. The computer-implemented method of claim 10, further comprising:
training, by the device, the first AI model to generate the first data by employing the second data and the third data as input data to the first AI model and employing known labor and delivery predictions corresponding to the input data as output data for the first AI model; and
training, by the device, the second AI model by employing a supervised machine learning process to identify patterns in training cardiotocograph data that correspond to defined physiological events associated with respective fetuses and mothers of the fetuses represented in the training cardiotocograph data.

16. The computer-implemented method of claim 10, further comprising:
generating, by the device, an alert in response to the first data being indicative of an emergency situation.

17. The computer-implemented method of claim 10, further comprising:
displaying, by the device, the first data at a device accessible to an entity for further analysis of the first data to identify actions to be executed by the entity for safe delivery of the one or more fetuses.

18. A computer program product for AI-based clinical decision support for labor and delivery, the computer program product comprising a non-transitory computer readable memory having program instructions embodied therewith, the program instructions executable by a processor to cause the processor to:
generate, during labor, first data comprising one or more labor and delivery predictions applicable to one or more fetuses and a mother of the one or more fetuses by analyzing, via a first AI model executed by the processor, second data comprising CTG analysis data of the one or more fetuses and the mother generated by a second AI model and third data comprising maternal health analysis data of the mother generated by a third AI model, wherein the first AI model is a multistage AI model comprising respective models directed to predicting respective ones of the one or more labor and delivery predictions.

19. The computer program product of claim 18, wherein the first data comprises one or more types of data selected from a group comprising fetal hypoxia, fetal acidemia, fetal acidosis, labor progression indicating a C-section, cervical dilation progression, a postpartum hemorrhage risk assessment, a preeclampsia prediction, a labor induction recommendation, a sepsis possibility, and one or more additional labor and delivery predictions.

20. The computer program product of claim 18, wherein the program instructions are further executable by the processor to cause the processor to:
generate the second data by processing FHR data of the one or more fetuses and UA data of the mother, and wherein the second data comprises one or more types of data selected from a group consisting of an FHR baseline value calculation, an FHR acceleration, an FHR deceleration, a contraction, an FHR variability value calculation, fetal tracing classification, and one or more additional CTG analysis data types; and
generate the third data by employing rule-based algorithms to process EMR and health parameters of the mother, wherein the third data comprises one or more types of data selected from a group consisting of maternal health related risk factors, pregnancy related complications, dystocia, genetic disorders and one or more additional maternal health analysis data types.
